(19) United States
(12) Patent Application Publication
(10) Pub. No.: US 2017/0051333 A1
(43) Pub. Date: Feb. 23, 2017

(54) COMPOSITIONS FOR CELL LYSIS AND USES THEREOF

(71) Applicant: Uwe Warnken, Frankfurt (DE)

(72) Inventor: Uwe Warnken, Frankfurt (DE)

(21) Appl. No.: 15/114,741

(22) PCT Filed: Jan. 26, 2015
(86) PCT No.: PCT/EP2015/051523

§ 371 Date: Jul. 27, 2016

(30) Foreign Application Priority Data
Jan. 27, 2014 (GB) 1401373.4

(51) Int. Cl. C12Q 1/68

(52) U.S. Cl. CPC C12Q 1/6806

(57) ABSTRACT

The present invention relates to a simple and highly efficient method for the isolation of nucleic acids from a sample, methods of lysing samples from which the nucleic should be obtained, and materials that can be used in such methods.

### TECHNICAL FIELD OF THE INVENTION

The present invention relates to methods for the processing of biological samples, particularly processes for the isolation of nucleic acids, such as RNA. The invention further provides products that can be used in such methods.

### BACKGROUND OF THE INVENTION

In recent years, molecular biology is increasingly used in clinical research, clinical diagnostic testing and drug discovery. In particular, methods based on the nucleic acid analysis, i.e. DNA and RNA have been developed. The provision of substantially pure and non-degraded nucleic acids is of central importance in such methods. In particular, RNA is prone to degradation due to RNases which are ubiquitous enzymes present in virtually all cellular environments.

Known methods for extracting RNA begin with one of a variety of techniques to disrupt or lyse cells, liberate RNA into solution, and protect RNA from degradation by endogenous RNases. Lysis liberates RNA along with DNA and protein from which the RNA must then be separated. Thereafter, the RNA is treated either to solubilize it or to precipitate it.

Chaotropic guanidinium salts are frequently used to simultaneously lyse cells, solubilize RNA and inhibit RNases a S disclosed in Chirgwin et al, Biochem., 18, 5294-5299 (1979). Other methods separate solubilized RNA from protein and DNA by extraction with phenol/chloroform at low pH (D. M. Wallace, Meth. Enzym., 15, 33-41 (1987)). One-step isolation methods to obtain RNA involve treating cells sequentially with 4 M guanidinium salt, sodium acetate (pH 4), phenol, and chloroform/isoamyl alcohol. Subsequently, samples are centrifuged and RNA is precipitated from the upper layer by the addition of alcohol (P.

A method in which a stable mixture of phenol and guanidinium salt at an acidic pH is added to the cells is described in U.S. Pat. No. 4,843,155. After phase separation with chloroform, the RNA in the aqueous phase is recovered by precipitation with an alcohol.

Other methods include adding hot phenol to a cell suspension, followed by alcohol precipitation (T. Maniatis et al, Molecular Cloning, A Laboratory Manual, Cold Spring Harbor Laboratory (1982)); the use of anionic or non-ionic surfactants to lyse cells and liberate cytoplasmic RNA; and the use of inhibitors of RNases such as vanadyl riboside complexes and diethylpyrocarbonate (L. G. Davis et al, “Guanidine Isothiocyanate Preparation of Total RNA” and “RNA Preparation: Mini Method” in Basic Methods in Molecular Biology, Elsevier, New York, pp. 130-138 (1991)).

A technique for isolating both DNA and RNA from biological sources by binding on glass or other solid phases was disclosed in U.S. Pat. No. 5,234,809 (Boom et al.). Cells present in biological sources, such as serum or urine, were lysed by exposure to strong (>5 M) solutions of guanidinium thiocyanate in Tris HCl (pH 8.0), containing EDTA and the surfactant Triton X-100. DNA and RNA were purified from biological materials by incubation with diatomaceous earth or silica particles, which formed reversible complexes with the DNA and RNA.

U.S. Pat. No. 6,355,792 discloses a method for isolating nucleic acids by acidifying a liquid sample with a buffer having a pH less than 6.5 and contacting the acidic solution with an inorganic oxide material having hydroxyl groups, separating the solid material with bound nucleic acids on it from the liquid, and eluting with alkaline solution having a pH between 7.5 and 11, preferably 8-8.5. The acidic solution is free of ionic detergents, chaotropes and any ions are <0.2 M.

EP 0707077 B1 discloses a method for providing a nucleic acid from a lysate comprising the steps of: contacting a lysate suspected of containing a nucleic acid at a pH of ≦7.0 with a water-soluble, weakly basic polymer in an amount sufficient to form a water-insoluble precipitate of said weakly basic polymer with all nucleic acids present in said lysate, separating said water-insoluble precipitate from said lysate, and contacting said precipitate with a base to raise the solution pH to ≧7, and thereby releasing said nucleic acids from said weakly basic polymer.

U.S. Pat. No. 5,973,137 discloses a method for isolating substantially non-degraded RNA from a biological sample by treating the sample with a cell lysis reagent consists of an anionic detergent, a chelating agent and a buffer solution having a pH less than 6. The role of the anionic detergent is said to lyse cells and/or solubilize proteins and lipids as well as to denature proteins.

U.S. Pat. No. 6,737,235 discloses a method for isolating nucleic acids using particles comprising or coated with a hydrophilic, cross-linked polyacrylamide polymer containing cationic groups. Cationic groups are formed by protonation at low pH of amine groups on the polymer. Nucleic acids are bound in a low ionic strength buffer at low pH and released in a higher ionic strength buffer. The polymers must have a lower critical solubility temperature of 25-45° C. Desorption is also promoted at alkaline pH and higher temperatures.

U.S. Pat. No. 6,875,857 discloses a method and reagent for isolating RNA from plant material using the reagent composition comprising the nonionic surfactant IGEPAL, EDTA, the anionic surfactant SDS, and a high concentration of 2-mercaptoethanol.

Several patents and applications disclose the reversible capture of nucleic acids onto binding materials mediated by pH change between binding and elution solutions changing the state of protonation of amine groups on the binding materials, e.g. EP 1036082 B1.

The multi-step nature of the prior methods for isolating nucleic acids such as RNA complicates the use of RNA in clinical practice. Methods must overcome the difficulty of separating RNA from the protein and DNA in the cell before the RNA is degraded by nucleases, such as RNase. These nucleases are e.g. present in blood in sufficient quantities to destroy non-protected RNA rapidly. Successful methods for the isolation of RNA from cells must therefore be capable of preventing degradation by RNases.

There remains a need in the art for a rapid, simple method for extracting RNA from biological samples. Such method would minimize hydrolysis and degradation of the RNA so that it can be used in various analyses and downstream processes. Further, there is a need for methods for the isolation and purification of nucleic acids from samples wherein cell lysis and protection of RNA and/or DNA are enabled through the use of a single agent that is not a chaotropic salt.

There is also a need to isolate sufficient quantities of essentially non-degraded nucleic acids, e.g. DNA, but in particular RNA, such as mRNA or microRNA from a given biological sample, e.g. a clinical specimen.

Still further, there is a need to provide methods and tools for the isolation of essentially intact, non-degraded nucleic acids in sufficient quantity for further analysis, wherein the nucleic acids are obtained from a sample that contains substances that could interfere with subsequent analysis, e.g. full-blood samples or stool samples.

Various suppliers of laboratory and clinical materials provide extraction kits for nucleic acids, e.g. for DNA, RNA as well as sub-types thereof which make the isolation of these target molecules relatively easy. However, even though the methods and kits currently on the market permit the isolation of nucleic acids from various sources, the integrity and quantity of nucleic acids, e.g. RNA, obtained from a given sample are frequently not satisfactory. Moreover, methods of the state of the art rely frequently on the use of different buffer systems and matrices such as silica, which are only optional in context of the present invention. Although these methods are relatively straightforward and can be performed quickly, obtaining RNA, e.g. microRNA, that are substantially pure and non-degraded can be difficult.

Accordingly, it is an object of the present invention to provide a simple and reliable method for isolating nucleic acids from a sample that is fast, economical and easy to perform, that avoids the need for large amounts of materials (e.g. laboratory material), that requires only a small number of manipulation steps through laboratory staff before the nucleic acids, e.g. RNA, is analysed, and which provides essentially pure and non-degraded nucleic acids.

A further objective underlying the work resulting in the present invention is to avoid the addition of enzymes to biological samples in order to digest these samples prior to the isolation of the nucleic acids (e.g. the use of proteinase K). Another objective is to provide methods allowing for the isolation of nucleic acids from a sample that can be in different states, e.g. a fresh sample, a fresh frozen sample, a snap-frozen sample, a formalin fixed sample, etc., or from sample per se not containing large amounts of nucleic acids, for example, single cell samples.

Yet another objective of the present invention is to avoid the use of chaotropic salts, detergents, urea, phenol and RNase inhibitors. These and other objectives are accomplished by the invention described below.

### FIGURES

FIG. 1: LDS 1D-PAGE analysis of proteins derived from HeLa cells lysed with HFIP spiked with 5 wt.-% ammonium acetate and 10 Vol.-% (v:v) water. After centrifugation of the lysed cell material, cellular proteins found in the liquid phase were almost completely dissolved in the lytic composition (Lane 1). Only traces of protein carryover also visible as high abundant protein bands in the supernatant lane were detectable in the HFIP pellet (Lane 2).

FIG. 2: 1D-PAGE analysis of the HeLa membrane proteome obtained according to the protocol of the Proteo Extract Subcellular Proteome Extraction Kit (Calbiochem) solubilized with either HFIP lysis buffer or conventional RIPA lysis buffer. 40 μg Proteo Extract Kit membrane proteome fraction from 10⁷ HeLa cells were either solubilized with HFIP containing 5 wt.-% ammonium acetate and 10 Vol.-% water (v:v) (Lane 1), or in RIPA lysis buffer (Lane 2). Proteomes displayed the same complexity and intensity in their 1D-PAGE pattern.

FIG. 3A-C: Bioanalyzer RNA chip analysis of three RNA samples of HeLa cells treated with HFIP lysis compositions containing 0.25 wt.-%, or 2.5 wt.-% or 5 wt.-% ammonium acetate. The obtained RNA was intact with no detectable signs of degradation, as determined by quantification of ribosomal 18S and 28S RNA ratios which were 9.9, 10.0 and 10.0 RIN (RNA integrity factor was used as parameter).

FIG. 4A-E: Bioanalyzer DNA chip analysis of nucleic acids isolated from 50 mg frozen HeLa cells were subjected to 500 ml HFIP composition comprising 2 wt-% ammonium acetate spiked with different quantities of water, 0 Vol.-%, 2.5 Vol.-%, 5 Vol.-%, 10 Vol.-% and 20 Vol.-% (each, v:v). HFIP separated RNA is substantially free of DNA. Nucleic acids were separated and subjected to RNase. DNA concentration was analyzed utilizing the Bioanalyzer 2100 and DNA LabChip 25-1000 bp.

FIG. 5A-D: Bioanalyzer RNA chip quality control of HFIP extracted total RNA samples intended for RT-PCR analysis (Agilent 2100 Bioanalyzer Eukaryote total RNA nano-chip analysis). RIN factor 10 was measured for each of RNA samples 1, 2 and 3 comprising total RNA extracted from HeLa cells and 9.8 for Super Script HeLa RNA control. According to these results, high yields of pure and intact total RNA samples are delivered with the HFIP lysis buffer.

FIG. 6: RT-PCR amplifications of RNA samples 1, 2 and 3 of total RNA extracted from HeLa cells provided with HFIP lysis buffer were performed utilizing 50 ng of cDNA and 26 pmol primers for DNA replication licensing factor-5 (MC1V15_Exon8), glyceraldehyde-3-phosphate dehydrogenase-1 and -2 (GAPDH1_Exon8, GAPDH2_Exon8). PCR products were analyzed by agarose gel electrophoresis and ethidium bromide staining to visualize the PCR amplification products.

### SUMMARY OF THE INVENTION

In the context of studies aiming at providing solutions to the above objectives, it was surprisingly found out that a fast and simple method for the selective enrichment of pure, intact cellular total nucleic acids, e.g. RNA, can be provided when the compositions according to the present invention are used, which comprise a large quantity of 1,1,1,3,3,3 hexafluoro-2-propanol (HFIP). It is unexpected that this method allows the provision of, e.g., small (21-28 nucleotides) to large (more than 200 nucleotides long) coding and non-coding forms of RNA from different sources, e.g. from humans, other animals, plants, microorganisms (e.g. bacteria or viruses).

The present invention further provides reagents and methods suitable for isolating nucleic acids, in particular RNA, from a biological sample wherein the obtained RNA is substantially free of DNA and protein. The obtained nucleic acids, particularly, RNA is substantially pure and suitable for gene expression analysis in research, clinical diagnosis, and other applications, e.g. reverse transcriptase polymerase chain reaction (RT-PCR). When RNA is separated after lysis of the cell sample following the steps disclosed herein below, the remainder of the cell sample, which is present in the suspension composed of the biological sample and the composition of the invention, may be further processed in order to isolate the proteins present therein for respective analysis. Similarly, DNA may be isolated after lysis of the cell sample using appropriate means.

In the inventive methods, a biological sample is treated with a concentrated organic solution of at most 20% (v:v), preferably about 5-15% (v:v) water in HFIP spiked with low amounts of dissolution modifiers, e.g. salts, preferably 0.1-5% (m:m) ammonium acetate, organic/inorganic acids, and organic/inorganic bases. Ammonium acetate is preferred for cell lysis with HFIP, because it is soluble in alcohols in general and therefore, easily removable from RNA, DNA and protein precipitates by washing with e.g. ethanol, 2-propanol etc. Ammonium acetate is volatile under low pressure and low amounts could be evaporated from biological samples. Water solutions of ammonium acetate are at neutral pH of 7, beneficial for further biological sample processing and analysis. The sample is lysed completely with this organic solvent composition and total RNA can be recovered. The quantity and quality of the obtained nucleic acid, e.g. RNA, is even better when HFIP compositions provided herein contain a low amount of an organic salt, and optionally a small volume of water. The addition of chaotropic salts, detergents or phenol is not necessary and not desired. Alternatively, lysis of cells may be conducted with an aqueous solution, e.g. water, comprising about 30% (v:v) HFIP, e.g. in a pressure cycling process, and thereafter the obtained cell lysate is treated with pure HFIP or a concentrated HFIP solution—spiked with low amounts of dissolution modifiers, e.g. salts, organic/inorganic acids, and organic/inorganic bases to reach a final proportion of at most 20% (v:v) water in HFIP. In a further alternative, cells are lysed with detergents, e.g. SDS, LDS, RIPA, Tween, Triton, Brij, CHAPS, CHAPSO etc., or chaotropic agents e.g. urea, guanidinium chloride, guanidinium thiocyanate etc. or Tris buffer and the like, and the lysate is treated with the above mentioned pure ore concentrated organic solution spiked with low amounts of dissolution modifiers, e.g. salts, organic/inorganic acids, and organic/inorganic bases to reach a final proportion of at most 20% (v:v) water in HFIP. In a further alternative, cells are lysed with detergents or chaotropic salts, e.g. SDS, LDS, guanidinium chloride, guanidinium thiocyanate, RIPA, Tris and the like, and the macromolecular components of the lysate is precipitatet with an excess of organic solvent comprising e.g. ethanol, 2-propanol, acetone, acetonitrile etc. After separation of the precipitate e.g. by centrifugation, the protein/DNA/RNA pellet is treated with the above mentioned pure or concentrated organic solution spiked with low amounts of dissolution modifiers, e.g. salts, organic/inorganic acids, and organic/inorganic bases to reach a final proportion of at most 20% (v:v) water in HFIP for protein lysis and nucleic acid separation. In a further application, biological samples, e.g. blood, sputum, tear fluid etc. is treated with the above mentioned pure ore concentrated organic solution spiked with low amounts of dissolution modifiers, e.g. salts, organic/inorganic acids, and organic/inorganic bases to reach a final proportion of at most 20% (v:v) water in HFIP. In an alternative application, biological samples, e.g. blood, sputum, tear fluid etc. is treated with an excess of organic solvent comprising e.g. ethanol, 2-propanol, acetone, acetonitrile etc. for precipitating DNA, RNA and protein. After separation of the precipitate e.g. by centrifugation, the protein/DNA/RNA pellet is treated with the above mentioned pure or concentrated organic solution spiked with low amounts of dissolution modifiers, e.g. salts, organic/inorganic acids, and organic/inorganic bases to reach a final proportion of at most 20 Vol,-% (v:v) water in HFIP for DNA and RNA separation.

In a further aspect of the invention, proteinaceous material (e.g. supernatants) obtained after any of the above mentioned methods for cell lysis may be supplemented with aqueous solutions of detergents or chaotropic agents, HFIP is evaporated and proteins solubilized in the detergent mixture (optionally after additional purification steps) may be used for protein analysis.

Concentrated HFIP is a known solvent for peptides and proteins. An unexpected result of the work underlying the present invention is that concentrated HFIP solutions are suitable for cell lysis and that total cellular proteomes could be completely dissolved in concentrated HFIP solutions spiked with low amounts of solubilizers. The low boiling point of about 58° C. is an advantage of using HFIP as solvent for cell lysis because higher temperatures could damage biomolecules, e.g. proteins or nucleic acids. HFIP evaporates readily from separated cellular components.

Hexafluoro-2-propanol is further known as solvent for some polar polymers and organic synthesis and particular in peptide synthesis. It is especially effective in the solubilization of polymers, including those that are rather insoluble in the most common organic solvents, e.g. polyamides, polyacrylonitriles, polyacetals, polyesters (e.g. polyglycolide), and polyketones. HFIP is used in biochemistry in solubilization of peptides (e.g. Narita et al., 1988) or in the monomerization of β-sheet protein aggregates (Pachahara et al, 2012). Due to its acidity (pKa=9.3), HFIP can be used as acid in volatile buffers for ion pair HPLC—mass spectrometry of nucleic acids.

The acidity of HFIP is caused by the strong electro-negative effect of six fluorine atoms symmetrically arranged around the secondary alcohol group. Accordingly, HFIP acts like a weak acid and dissociates in aqueous solutions.

Up to 30% HFIP in water acts as an enhancer of protein solubilization, particularly in combination with other protein solubilizers like urea (Gross et al., 2008). Such formulations are used in the lysis of cells in Pressure Cycling Technology (Lazarev et al.). Herein, lysis of cells derived from cell culture, tissues or organs is effected by strong pressure in specially developed lysis tubes with HFIP containing agent.

One reason for the remarkable dissolving power of HFIP is a high dipole moment. Dependent on the steric structure of the molecules, dipole moments of HFIP molecules vary in a range from 0.64-3.17 Debye. The dipole moment of water is 1.85 D at 20° C. Solvents with strong hydrogen-bonding capabilities are good solubilizers for ionic-, polar- and polarizable compounds.

Cell lysis with pure HFIP is generally not desirable, because pure HFIP denatures proteins to a substantial extent, and as a consequence, formation of insoluble undefined poly-electrolyte aggregates of protein, DNA and RNA can occur. These insoluble aggregates “shield” RNA present therein. RNA complexed by these aggregate shields is difficult to solubilize in water and may be lost for further analysis. It was surprising to notice that ionic substances such as inorganic or organic acids, inorganic or organic bases, or organic salts of fatty acids have a positive impact on the strong protein solvatisation ability of HFIP. This effect makes compositions comprising high concentrations of HFIP suitable in the provision of both, proteinaceous material and nucleic acids derived from biological samples for further specific analysis.

To reduce the acidity of HFIP and to reach pH values allowing the use of respective highly-concentrated HFIP based compositions for further biochemical analysis ionic substances such as inorganic or organic acids, inorganic or organic bases, or organic salts of fatty acids can be added to high-concentrated HFIP solutions. Complex cellular protein mixtures can be dissolved completely and protein aggregation with DNA/RNA is not observed following cell lysis. Therefore, it is even possible to solubilize protein/protein and protein/DNA/RNA aggregates and separate the fractions when the compositions of the present invention are used.

An explanation for the enhanced protein solubilization capacity of HFIP spiked with salts could be that the solvation of ions leads to higher ordered structures of HFIP towards an optimal steric adjustment of HFIP molecules for strong hydrogen-bonding. These effects can be used also in methods of the present invention which provide surprisingly high amounts of nucleic acids of outstanding quality, which can subjected to further biochemical analysis.

Accordingly, the present invention relates, inter alia, to the following aspects:
1. A composition comprising:
   (a) 1,1,1,3,3,3, hexafluoro-2-propanol (HFIP), and
   (b) a salt, and/or
   (c) an acid, and/or
   (d) a base, and/or
   (e) water, and/or
   (f) optionally a short chained branched or unbranched alcohol or ether or cyclic ether.
   The optionally present short chained branched or unbranched alcohol or ether or cyclic ether improves RNA precipitation by lowering the density of the HFIP lysis buffer, enhancing the hydrophobicity of the solution and perhaps, reducing the H-bonding strength of HFIP clusters.
2. The composition according to item 1, comprising at least 70% (v:v), more preferably at least 75% (v:v), still more preferred at least 80% (v:v) HFIP.
3. The composition according to item 1 or item 2, wherein the salt is selected from the group comprising ammonium, sodium, potassium, lithium, manganese, and barium, or the salt is a salt of an organic acid, e.g. formic acid, acetic acid, propionic acid, long chain fatty acids, di-carbonic acids, e.g. oxalic acid, malonic acid, succinic acid, or citric acid.
4. The composition according to any one of items 1 to 3, wherein the organic salt, the organic acid, inorganic acid, organic base, and/or inorganic base is added to the HFIP in an amount of 0.05 weight-% up to saturation level.
5. The composition according to any one of items 1 to 4 comprising water in an amount of 1.0 to 20.0 Vol.-%.
6. The composition according to any one of items 1 to 5 for use as a lysis agent for biological samples.
7. The composition according to any one of items 1 to 6 for use in the extraction of nucleic acids from a cell sample.
8. A method of extracting nucleic acids from a sample comprising the steps:
   (a) providing a cell sample,
   (b) adding a composition according to any one of items 1 to 5 to said sample,
   (c) suspending the cell sample in said composition,
   (d) separating nucleic acids,
   (e) dissolving nucleic acids in an aqueous buffer.
9. The method of item 8, wherein the separation of nucleic acids is performed by centrifugation of the suspension and precipitation of the nucleic acids to provide a supernatant, and a nucleic acid sediment, or by filtration of the nucleic acids, e.g. through use of special RNA-binding glass-fiber filter cartridges for RNA or DNA separation, which are suitable for vacuum filtration or centrifugation methods, or through use of polyethylene or any other filter cartridge for particle or sediment filtration, through use of molecular sieves or any chromatographic devices.
10. The method of any one of items 7 to 9, further comprising the addition of aliphatic alcohols selected from the group comprising methanol, ethanol, 1-propanol, 2-propanol, n-butanol, isobutanol, and 2-butanol, isomers of amyl alcohol, trifluoroethanol, or esters of aliphatic alcohols and fatty acids selected from the group comprising, methylacetate, ethylacetate, or ketones selected from the group dimethyl ketone, methyl ethyl ketone, or ethers selected from the group comprising diethyl ether, 1,2-, 1,3-, 1,4-dioxane, or other organic solvents comprising acetonitrile, and chloroform prior to or after step (b).
11. The method of any one of item 10, wherein aliphatic alcohols selected from the group comprising methanol, ethanol, 1-propanol, 2-propanol, n-butanol, isobutanol, and 2-butanol, isomers of amyl alcohol, trifluoroethanol, or esters of aliphatic alcohols and fatty acids selected from the group comprising methylacetate, ethylacetate, or ketones selected from the group dimethyl ketone, methyl ethyl ketone, or ethers selected from the group comprising diethyl ether, 1,2-, 1,3-, 1,4-dioxane, or other organic solvents comprising acetonitrile, and chloroform is added in an amount of 0.1 to 25.0 Vol.-% of the suspension.
12. The method of any one of item 10, wherein further a nucleic acid binding material is added to the suspension obtained in step (b).
13. The method of any one of items 7 to 12, wherein the supernatant is removed by decanting, aspiration or evaporation.
14. The method of any one of items 7 to 13, wherein the nucleic acid sediment is washed with a solution comprising at least 70% (v:v) HFIP, at least 75% (v:v) HFIP, at least 80% (v:v) HFIP, preferably at least 90% (v:v) HFIP, more preferably at least 95% (v:v) HFIP, or 99% (v:v) HFIP, wherein said washing solution is removed after the washing step.
15. The method of any one of items 7 to 14, wherein the nucleic acid is RNA.
16. The method of any one of items 7 to 15, wherein the nucleic acid is mRNA or microRNA.
17. The method of any one of items 7 to 16, wherein the RNA obtained in step (d) is dissolved in a suitable medium, e.g. sterilized water or DEPC treated water.
18. The method of any one of items 7 to 17, wherein the RNA is subjected to reverse transcription.
19. The method of any one of items 7 to 18, wherein the nucleic acid is subjected to amplification, hybridization, or sequencing.
20. A kit for the lysis of a cells present in a sample comprising a composition according any one of items 1 to 5 and instructions for use.
21. The kit according to item 20 further comprising aliphatic alcohols selected from the group comprising methanol, ethanol, 1-propanol, 2-propanol, n-butanol, isobutanol, and 2-butanol, isomers of amyl alcohol, trifluoroethanol, or esters of aliphatic alcohols and fatty acids selected from the group comprising methylacetate, ethylacetate, or ketones selected from the group dimethyl ketone, methyl ethyl ketone, or ethers selected from the group comprising diethyl ether, 1,2-, 1,3-, 1,4-dioxane, or other organic solvents comprising acetonitrile, and chloroform etc. and/or a nucleic acid binding material.

### DEFINITIONS

Before describing the invention in more detail some terms and expressions used hereinafter are explained.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will be decisive.

As used in the context of present invention, the singular forms of “a” and “an” also include the respective plurals unless the context clearly dictates otherwise.

The term “about” in context with a numerical value or parameter range denotes an interval of accuracy that the person skilled in the art will understand to still ensure the technical effect of the feature in question. The term typically indicates deviation from the indicated numerical value of +/−10%, preferably +/−5%.

The term “nucleic acid” designates a sequence of nucleic acid building blocks, such as deoxyribonucleic acid, ribonucleic acid, and/or chemical analogs thereof, i.e. molecules, which may replace building blocks in nucleic acids, such as PNA molecules, LNA molecules, etc. Nucleic acids may be single stranded or double stranded, or may contain portions of both double stranded and single stranded sequences. The nucleic acid may be DNA, RNA, or a hybrid, where the nucleic acid may contain combinations of deoxyribo- and ribonucleotides, and combinations of bases including uracil, adenine, thymine, cytosine, guanine, inosine, xanthine hypoxanthine, isocytosine and isoguanine. Nucleic acid refers to nucleic acid found within cellular material and can be genomic DNA and RNA, and other nucleic acids such as that from infectious materials, including viruses and plasmids.

The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The terms “extracted” or “extraction” mean that a compound or group of compounds, e.g. nucleic acids are “extracted or purified from a cell or organism of origin”.

As used herein, the term “miRNA” or “microRNA” refers to an RNA, preferably a single-stranded RNA, of about 10-50 nucleotides in length (the term “nucleotides” including nucleotide analogs), preferably between about 10-30 nucleotides in length, e.g. about 12, 13, 14, 15, 16, 17, 18, 19, 20, 21, 22, 23, 24, 25, 26, 27, or 28 nucleotides in length. MicroRNAs (miRNAs) are important molecules that seem to be involved in eukaryotic RNA silencing mechanism known as RNA interference (RNAi). Numerous recent studies have investigated the role of miRNAs in regulating gene expression. Apparently, miRNAs “fine-tune” gene expression by binding to nearly perfect complementary sequences in mRNAs, thus preventing their translation. The importance of miRNAs in the regulation of specific genes has been demonstrated in a variety of organisms, where their function impacts such universal cellular pathways as cell death, development, proliferation, and hematopoiesis (Ambros, 2004).

“Biological sample” or “cell sample” or “sample” as used herein may mean a sample of biological tissue, fluid, or surface (e.g. skin, mucosa, etc.) that comprises nucleic acids. Such samples include, but are not limited to, tissue isolated from plants or animals, preferably mammals, such as humans. Biological samples may also include sections of tissues such as biopsy and autopsy samples, frozen sections taken for histological purposes, blood, plasma, serum, sputum, stool, tears, mucus, hair, and skin. Biological samples also include explants and primary and/or transformed cell cultures derived from patient tissues. A sample may be provided by removing a sample of cells from an animal, but can also be accomplished by using previously isolated cells (e.g., isolated by another person, at another time, and/or for another purpose), or by performing the methods of the invention in vivo. Archival tissues, such as those having treatment or outcome history, may also be used. Furthermore, a sample may be obtained from the environment, i.e. environmental biological samples can be derived from water, soil, air, food, agricultural products, devices, stables, laboratory surfaces, surfaces of publically accessible buildings or premises, such as hospitals, swimming pools, toilets, restaurants, and the like.

In the context of the present invention, a “microorganism” designates viruses, bacteria, fungi, parasites, etc. The microorganism can be a pathogen, e.g. a pathogen for mammals, such as humans

The term “mRNA” or “messenger RNA” refers to a single-stranded RNA that specifies the amino acid sequence of one or more polypeptide chains. This information is translated during protein synthesis when ribosomes bind to the mRNA.

The terms “release”, “elute” means that a substantial portion of a material bound to the surface or pores of a solid phase material is removed by contact with a solution or composition.

The term “nucleic acid binding material” or “matrix” designates a material having a surface which can attract nucleic acid molecules. These materials can be in the form of particles, microparticles, nanoparticles, fibers, beads, membranes, filters and other supports such as test tubes and microwells. The particles can be, e.g. particles silicium dioxide.

As used herein, the term “salt”, which may be present in a composition of the present invention is preferably selected from ammonium-, lithium-, sodium-, potassium-salts, e.g. ammonium acetate, sodium acetate, and so forth.

As used herein, the term “acid” generally refers to any aqueous solution having a pH below neutral pH. Preferably the solution will have a pH in the range of 1-5 and more preferably from about 2-4. The acid can be organic or inorganic. Mineral acids such as hydrochloric acid, sulfuric acid, and perchloric acid are useful. Organic acids including monocarboxylic acids, dicarboxylic acids, tricarboxylic acids, and amino acids can be used, as well as salts of the acids. Representative acids include formic, acetic, trifluoroacetic, propionic, oxalic, malonic, succinic, glutaric, and citric acids, glycine, and alanine. Salts can have any water-soluble counter ion, preferably alkali metal or alkaline earth ions (e.g. preferably Mg or Ba ions, most preferably Mg-acetate or Ba-acetate) or ammonium ions. Acidic solutions comprising salts of transition metals are also useful in the practice of the present invention. Preferred transition metals include Fe, Mn, Co, Cu, and Zn salts, e.g. FeCl₃×10 H₂O.

As used herein, the term “base” generally refers to any aqueous solution having a pH above neutral pH. Inorganic bases like sodium- and potassium—hydroxide, barium hydroxide and ammonia, which are well soluble in HFIP and neutralize HFIP in an acid-base reaction to provide soluble hexafluoropropanol-alcoholates, are suitable to improve solubilization of proteins in HFIP.

As used herein, the term “water” generally refers to sterilized or DEPC-treated water. Water should always be free of RNases or other organic contamination. DEPC treated water is preferably used in the compositions, or methods, kits of the present invention.

As used herein, the term “amplification” refers to the process of enzyme-mediated de novo synthesis of nucleic acid sequences, e.g. using enzymatic methods such as RT-PCR, PCR, qPCR, and the like, all of which are known to a person skilled in the art. In a further aspect of the invention, it is noted that the compositions of the present invention described above can be used for the purification of PCR products, DNA and RNA cloning products, and synthetic DNA and RNA products.

As used herein, the term “hybridization” generally refers to process of pairing or annealing of complementary nucleic acid sequences, e.g. using probes targeting and hybridizing with a given target nucleic acid, e.g. a nucleic acid that needs to be detected. Hybridization is a mechanism underlying methods such as Northern Blot, primer annealing to complementary nucleic acid strands in reverse transcription reactions, or in PCR reactions.

Hybridization may also be applied in methods such as In Situ Hybridization (ISH) or Fluorescence In Situ Hybridization (FISH) or single molecule Florescence In Situ Hybridisation (smFISH) or reverse transcriptase (RT) in situ PCR of DNA and RNA target transcripts annealing to complementary or partially complementary nucleic acid molecules that are detected with labeled probes.

As used herein, the term “sequencing” refers to the process of determining the sequence of nucleotide building blocks in a sample of interest, e.g. using conventional Maxim-Gilbert Sequencing or recently established, partially automatic or semi-automatic methods generally known as “Next Generation Sequencing” or “Massively parallel sequencing”.

As used herein, the term “highly concentrated HFIP composition” refers to compositions comprising a quantity of at least about 70% (v:v) of HFIP.

As used herein, the term “pure HFIP composition” refers to compositions comprising a quantity of at least 99% (v:v) of HFIP as delivered from the supplier.

As used herein, the expression “short chained branched or unbranched alcohol” refers to the group comprising methanol, ethanol, 1-propanol, 2-propanol, n-butanol, isobutanol, and 2-butanol, isomers of amyl alcohol, trifluoroethanol etc.

As used herein, the expression “ester” refers to the group comprising compounds of aliphatic alcohols and fatty acids selected from the group comprising, methylacetate, ethylacetate etc.

As used herein, the expression “ketone” refers to the group comprising dimethyl ketone, methyl ethyl ketone etc.

As used herein, the expression “ether” refers to the group comprising diethyl ether, fluoromethyl hexafluoroisopropyl ether, 1,2,2,2-tetrafluoroethyl difluoromethyl ether etc.

The expression “cyclic ether” refers to the group comprising 1,2-, 1,3-, 1,4-dioxane etc.

### DETAILED DESCRIPTION

The present invention is concerned with rapid and simple methods for obtaining nucleic acids, such as RNA from biological samples.

The compositions of the present invention are efficient in the lysis of cell membranes or cell walls due to their degrading effect on proteinaceous materials.

Degradation of nucleic acids is minimized because the RNA is released directly into the inventive composition, which is free of nucleases, in particular free of ribonucleases (RNases) by virtue of the protein degrading effect of HFIP.

Another advantage associated with the use of the methods or tools of the invention is that it is possible to recover ribonucleic acids from samples with RNase activity without requiring the addition of RNase-inactivating compounds or proteins, such as guanidinium salts, high concentration chaotropes or RNase-inhibiting proteins and antibodies.

Methods of the invention are useful to capture and extract RNA from protein-RNA complexes, intact cells and viruses. RNA can be extracted according to the process of the invention from any biological sample containing nucleic acids, in particular intact cells of multicellular organisms or unicellular organisms, e.g. microorganisms (including viruses). Common sources of biological samples include, but are not limited to, bacterial culture or pellets, blood, urine, cells, bodily fluids such as urine, sputum, semen, CSF, blood, plasma, and serum, or tissues, e.g. biopsy materials.

The method of the invention can be applied to samples including viable, dead, or apoptotic intact cells and tissues, or cultured bacterial, plant or animal cells or cell lines without the need to subject them to other preliminary procedures. In particular, no preliminary disruption or lysis is required, e.g. for animal cell lines. When a pretreatment with other conventional lytic agents is necessary, e.g. for certain bacterial or plant cells, the lysed cells may subsequently be subjected to the methods of the invention.

The method of this invention is rapid, typically requiring only a few minutes to complete. Significantly, the RNA obtained by the method is of an adequate purity such that it is useful for clinical or other downstream uses, such as the use of reverse transcriptase, by itself or followed by the polymerase chain reaction amplification (RT-PCR), RNA blot analysis and in vitro translation.

Advantageously, it is not necessary to pretreat samples (e.g. with protein-degrading enzymes or other lytic agents) prior to use of this method and only simple equipment is required to perform the method. Detergents, urea or chaotropic substances for lysing biological samples are not needed or used. Due to the strong lytic activity, infectious agents, such as bacteria and viruses that might be present in the sample are degraded and their infectivity is essentially removed, with the exception of infectious nucleic acids found in some viruses.

The method is adaptable to automated platforms for processing large numbers of samples in serial or parallel fashion. The lysis, separation and washing steps are preferably performed for only a brief period, preferably for not more than 15, not more than 10 minutes, preferably not more than 5 minutes. Wash steps with specific buffers are frequently not necessary or can preferably be performed within a few minutes. Elution is preferably performed in not more than a few minutes.

The bound RNA is eluted from the test tube or from an optionally added matrix material by contacting the matrix material with a reagent to release the bound RNA into solution. The solution dissolves and sufficiently preserves the released RNA. RNA eluted in the release solution is compatible with downstream molecular biology processes.

The lysis step can be performed at room temperature (e.g. at 17-25° C.), but any convenient temperature can be used. It is also possible to perform the lysis step at 4° C. (e.g. in a cold room, optionally for an overnight incubation). Temperature for the elution of nucleic acids is not critical to the success of the present methods of isolating nucleic acids. Ambient temperature is preferred, but elevated temperatures may increase the rate of elution in some cases.

Kits are provided for performing the methods of the invention. A kit for isolating ribonucleic acid from a sample in accordance with the invention comprises at least one composition according to the invention, optionally also a matrix material selected to have the ability to bind nucleic acids released from biological samples into the suspension consisting of the inventive compositions and the sample material. Further, the kit may contain a container comprising a solution containing or consisting of aliphatic alcohols selected from the group comprising methanol, ethanol, 1-propanol, 2-propanol, n-butanol, isobutanol, and 2-butanol, isomers of amyl alcohol, trifluoroethanol, or esters of aliphatic alcohols and fatty acids selected from the group comprising methylacetate, ethylacetate, or ketones selected from the group dimethyl ketone, methyl ethyl ketone, or ethers selected from the group comprising diethyl ether, 1,2-, 1,3-, 1,4-dioxane, or other organic solvents comprising acetonitrile, and chloroform etc., or other alcohols, ketones, esters and ethers which can be added to the suspension of the inventive compositions and the sample material. Kits may additionally comprise an elution reagent, and one or more optional wash buffers and other conventional components of kits such as instruction manuals, protocols, buffers and diluents.

The invention also relates to compositions comprising (a) HFIP, and/or (b) a salt, and/or acid and/or a base, and/or (c) water. Optionally, the compositions may optionally comprise aliphatic alcohols selected from the group comprising methanol, ethanol, 1-propanol, 2-propanol, n-butanol, isobutanol, and 2-butanol, isomers of amyl alcohol, trifluoroethanol, or esters of aliphatic alcohols and fatty acids selected from the group comprising, methylacetate, ethylacetate, or ketones selected from the group dimethyl ketone, methyl ethyl ketone, or ethers selected from the group comprising diethyl ether, 1,2-, 1,3-, 1,4-dioxane, or other organic solvents comprising acetonitrile, and chloroform.

Further optionally, the compositions may comprise a matrix material that is suitable for binding nucleic acids, e.g. silicium dioxide in a suitable amount that is known to the person skilled in the art.

The compositions of the present invention preferably contain a high concentration of HFIP as set forth below:
a) The compositions of the present invention comprise more than about 50% of HFIP, or more than 55%, or more than 60%, or more than 65%, or more than 70%, or more than 75%, or more than 80%, or more than 85%, or more than 90%, or more than 91%, or more than 92%, or more than 93%, or more than 94%, or more than 95%, or more than 96%, or more than 97%, or more than 98%, or 99% HFIP.
b) Any one of the above compositions in (a) comprising more than 50% of HFIP, or more than 55%, or more than 60%, or more than 65%, or more than 70%, or more than 75%, or more than 80%, or more than 85%, or more than 90%, or more than 91%, or more than 92%, or more than 93%, or more than 94%, or more than 95%, or more than 96%, or more than 97%, or more than 98%, or 99% HFIP, may further comprise an ion source, e.g. an organic salt in an amount of 0.1 wt.-%, or in an amount of 0.25 wt.-%, or in an amount of 0.5 wt.-%, or in an amount of 0.75 wt.-%, or in an amount of 1.0 wt.-%, or in an amount of 1.25 wt.-%, or in an amount of 1.5 wt.-%, or in an amount of 1.75 wt.-%, or in an amount of 2.0 wt.-%, or in an amount of 2.5 wt.-%, or in an amount of 3.0 wt.-%, or in an amount of 4.0 wt.-%, or in an amount of 5.0 wt.-%, or in an amount of 6.0 wt.-%, or in an amount of 7.0 wt.-%, or in an amount of 7.5 wt.-%, or in an amount up to saturation.
c) Any of the above compositions of the present invention in sections (a) or (b) comprising more than about 50% of HFIP, or more than 55%, or more than 60%, or more than 65%, or more than 70%, or more than 75%, or more than 80%, or more than 85%, or more than 90%, or more than 91%, or more than 92%, or more than 93%, or more than 94%, or more than 95%, or more than 96%, or more than 97%, or more than 98%, or 99% HFIP, or the compositions comprising more than 50% of HFIP, or more than 55%, or more than 60%, or more than 65%, or more than 70%, or more than 75%, or more than 80%, or more than 85%, or more than 90%, or more than 91%, or more than 92%, or more than 93%, or more than 94%, or more than 95%, or more than 96%, or more than 97%, or more than 98%, or 99% HFIP, further comprising an organic salt in an amount of 0.1 wt.-%, or in an amount of 0.25 wt.-%, or in an amount of 0.5 wt.-%, or in an amount of 0.75 wt.-%, or in an amount of 1.0 wt.-%, or in an amount of 1.25 wt.-%, or in an amount of 1.5 wt.-%, or in an amount of 1.75 wt.-%, or in an amount of 2.0 wt.-%, or in an amount of 2.5 wt.-%, or in an amount of 3.0 wt.-%, or in an amount of 4.0 wt.-%, or in an amount of 5.0 wt.-%, or in an amount of 6.0 wt.-%, or in an amount of 7.0 wt.-%, or in an amount of 7.5 wt.-%, or in an amount up to saturation, wherein the salts in the compositions of the present invention may be selected from ammonium-, sodium-, or potassium salts, may further comprise an amount of 0.1 wt.-%, or an amount of 0.25 wt.-%, or an amount of 0.5 wt.-%, or an amount of 0.75 wt.-%, or an amount of 1.0 wt.-%, or an amount of 1.25 wt.-%, or an amount of 1.5 wt.-%, or an amount of 1.75 wt.-%, or an amount of 2.0 wt.-%, or an amount of 2.5 wt.-%, or an amount of 3.0 wt.-%, or an amount of 4.0 wt.-%, or an amount of 5.0 wt.-%, or an amount of 6.0 wt.-%, or an amount of 7.0 wt.-%, or an amount of 7.5 wt.-%, or an amount up to saturation of an organic or inorganic acid or an organic or inorganic base.
d) Further, any of the above compositions may further comprise aliphatic alcohols selected from the group comprising methanol, ethanol, 1-propanol, 2-propanol, n-butanol, isobutanol, and 2-butanol, isomers of amyl alcohol, trifluoroethanol, or esters of aliphatic alcohols and fatty acids selected from the group comprising methylacetate, ethylacetate, or ketones selected from the group dimethyl ketone, methyl ethyl ketone, or ethers selected from the group comprising diethyl ether, 1,2-, 1,3-, 1,4-dioxane, or other organic solvents comprising acetonitrile, and chloroform in any of the following amounts 0.1 Vol.-%, or 0.25 Vol.-%, or 0.5 Vol.-%, or 0.75 Vol.-%, or 1.0 Vol.-%, or 1.25 Vol.-%, or 1.5 Vol.-%, or 1.75 Vol.-%, or 2.0 Vol.-%, or 2.5 Vol.-%, or 3.0 Vol.-%, or 4.0 Vol.-%, or 5.0 Vol.-%, or 6.0 Vol.-%, or 7.0 Vol.-%, or 7.5 Vol.-%, or 10.0 Vol.-%, or 12.5 Vol.-%, or 15.0 Vol.-%, or 17.5 Vol.-%, or 20.0 Vol.-%.

Some exemplary and preferred compositions have the following contents:
(i) 95 wt.-% HFIP and 5 wt.-% of a salt (e.g. ammonium acetate);
(ii) 98 wt.-% HFIP and 2 wt.-% of a salt (e.g. ammonium acetate);
(iii) 97.5 wt.-% HFIP and 2.5 wt.-% of a salt (e.g. ammonium acetate);
(iv) 99 wt.-% HFIP and 1 wt.-% of a salt (e.g. ammonium acetate);
(v) 99.75 wt.-% HFIP and 0.25 wt.-% of a salt (e.g. ammonium acetate);
(vi) 99.5 wt.-% HFIP and 0.5 wt.-% of a salt (e.g. ammonium acetate);
(vii) 85 wt.-% HFIP, 5 wt.-% of a salt (e.g. ammonium acetate), and 10 wt.-% water;
(viii) 88 wt.-% HFIP, 2 wt.-% of a salt (e.g. ammonium acetate), and 10 wt.-% water;
(ix) 90 wt.-% HFIP, 5 wt.-% of a salt (e.g. ammonium acetate), and 5 wt.-% water;
(x) 92.5 wt.-% HFIP, 2.5 wt.-% of a salt (e.g. ammonium acetate), and 5 wt.-% water;
(xi) 90 wt.-% HFIP, 2.5 wt.-% of a salt (e.g. ammonium acetate), and 7.5 wt.-% water;
(xii) 93 wt.-% HFIP, 5 wt.-% of a salt (e.g. ammonium acetate), and 2 wt.-% water;
(xiii) 94 wt.-% HFIP, 5 wt.-% of a salt (e.g. ammonium acetate), and 1 wt.-% water;
(xiv) 80 wt.-% HFIP, 5 wt.-% of a salt (e.g. ammonium acetate), and 15 wt.-% water
(xv) 75 wt.-% HFIP, 5 wt.-% of a salt (e.g. ammonium acetate), and 20 wt.-% water
(xvi) 87.5 wt.-% HFIP, 2.5 wt.-% of a salt (e.g. ammonium acetate), and 10 wt.-% water;
(xvii) 86.5 wt.-% HFIP, 1.0 wt.-% of a salt (e.g. ammonium acetate), and 12.5 wt.-% water;
(xviii) 89.75 wt.-% HFIP, 0.25 wt.-% of a salt (e.g. ammonium acetate), and 10 wt.-% water;
(xix) 89.5 wt.-% HFIP, 0.5 wt.-% of a salt (e.g. ammonium acetate), and 10 wt.-% water;
(xx) 88.5 wt.-% HFIP, 1.5 wt.-% of a salt (e.g. ammonium acetate), and 10 wt.-% water.
(xxi) 87.5 wt.-% HFIP, 2.5 wt.-% of a salt (e.g. ammonium acetate), and 10 wt.-% water;
(xxii) 87 wt.-% HFIP, 3 wt.-% of a salt (e.g. ammonium acetate), and 10 wt.-% water;
(xxiii) 87 wt.-% HFIP, 3 wt.-% of a salt (e.g. ammonium acetate), and 10 wt.-% water;
(xxiv) 86 wt.-% HFIP, 4 wt.-% of a salt (e.g. ammonium acetate), and 10 wt.-% water;
(xxv) 80 wt.-% HFIP, 2.5 wt.-% of a salt (e.g. ammonium acetate), and 17.5 wt.-% water;
(xxvi) 77.5 wt.-% HFIP, 2.5 wt.-% of a salt (e.g. ammonium acetate), and 20 wt.-% water.

Further exemplary compositions have the following compositions shown in the table below.

| Example | HFIP | H₂O | Salt* | 2-propanol |
|---|---|---|---|---|
| No. | (vol.-% v:v) | (vol.-% v:v) | (wt.-%) | (vol.-% v:v) |
| 1 | 60 | 15.0 | 5.0 | 20.0 |
| 2 | 65 | 10.0 | 5.0 | 20.0 |
| 3 | 70 | 10.0 | 5.0 | 15.0 |
| 4 | 70 | 7.5 | 5.0 | 17.5 |
| 5 | 70 | 7.5 | 2.5 | 20.0 |
| 6 | 70 | 5.0 | 5.0 | 20.0 |
| 7 | 75 | 7.5 | 2.5 | 15.0 |
| 8 | 75 | 10.0 | 5.0 | 10.0 |
| 9 | 75 | 10.0 | 6.0 | 9.0 |
| 10 | 75 | 10.0 | 4.0 | 11.0 |

*e.g. NH₄C₂H₃O₂

Of course it is possible to slightly modify the above compositions so that up to 5.0% more or less of the respective ingredients can be used. Preferred compositions that may also be used according to the invention comprise about 10.0 (wt.-%) H₂O and about 5.0 (wt.-%) salt, e.g. ammonium acetate, and about 10.0 (wt.-%) of 2-propanol, the remainder being HFIP. These compositions can also be added directly into cell culture flasks upon aspiration of the culture medium. The contents of the cells will then be released (i.e. nucleic acids, etc.).

The above list of compositions is by no means complete. Further, it is possible to replace the salt by the corresponding amount of an organic or inorganic base or an organic or inorganic acid. It is possible also to use salt and an organic or inorganic base or an organic or inorganic acid in ratios of 1:1, 2:1, 1:2, 3:1, 1:3, 4:1, 1:4, 5:1, 1:5, 6:1, 1:6, 7:1, 1:7, 8:1, 1:8, 9:1, or 1:9 in any of the above exemplary compositions (i) to (xxvi). Preferably, the compositions have a pH of about 5.5 to 8.0, e.g. 6.5 to 7.5, e.g. about pH 7.0 for further biochemical applications of the obtained cellular components, i.e. proteins or nucleic acids.

In the methods of lysing cells and isolating nucleic acids, such as RNA, it is possible to add about 20 Vol.-%, of aliphatic alcohols selected from the group comprising methanol, ethanol, 1-propanol, preferably 2-propanol, n-butanol, isobutanol, and 2-butanol, isomers of amyl alcohol, trifluoroethanol, or esters of aliphatic alcohols and fatty acids selected from the group comprising methylacetate, ethylacetate, or ketones selected from the group dimethyl ketone, methyl ethyl ketone, or ethers selected from the group comprising diethyl ether, 1,2-, 1,3-. 1,4-dioxane, or other organic solvents comprising acetonitrile, and chloroform based on the volume of the suspension comprising the composition of the present invention and the biological sample.

Any of the above compositions in sections a) to d) and in the above examples (i) to (xxvi) and the compositions in the preceding paragraphs can be used as an agent for the lysis of cell samples.

Further, any of the above compositions in sections a) to d) and in the above examples (i) to (xxvi) and the compositions in the preceding paragraphs can be used as an agent in the extraction of nucleic acids, preferably in the extraction of RNA.

Further, none of the above compositions in sections a) to d) and in the above examples (i) to (xxvi) and the compositions in the preceding paragraphs that can be used as an agent in the extraction of nucleic acids, preferably in the extraction of RNA, or that is used as cell lysis agent contains either chaotropic salts, RNase inhibitors, other lytic or degrading enzymes, or detergent(s).

The present invention also provides methods for the lysis of cells in an obtained sample.

Further, the present invention provides methods and tools for the provision of protein fractions of obtained samples for further analysis.

Additionally, the present invention provides methods for the extraction, isolation and/or purification of nucleic acids. In one embodiment of such methods, the nucleic acids comprise RNA, e.g. mRNA, microRNA or other RNAs. In one embodiment, the nucleic acids comprise mRNA. In another embodiment, the nucleic acids comprise RNA, e.g. microRNA. It is also possible to reconstitute and use DNA, which was not eluted with RNA following the methods of the invention, and analyze the DNA further according to known methods.

In the methods for the extraction, isolation and/or purification of nucleic acids referred to above, any of the compositions set forth supra in can be used, e.g. the compositions set forth above in items (a) to (d) as exemplified by the compositions in (i) to (xxvi).

In the methods of the invention a biological sample, e.g. a cell sample, an individual cell, a clinical sample obtained from an animal, e.g. a mammal, preferably a human being, is provided. The sample is added to a composition of the invention as defined above. Incubation of the sample in the inventive composition causes the lysis of cells and the release of nucleic acids from the cytoplasm or nucleoplasm into the composition.

Depending on the sample size and the selected composition of the invention, the lytic process is completed within a few seconds (when the cell sample is small compared with the volume of the composition of the invention, i.e. when the sample-to-composition volume ratio is large) or several minutes, e.g. when the sample-to-composition volume ratio is small. The complete lysis of tissue samples or plant tissue samples usually takes longer than the complete lysis of e.g. fluid samples, such as blood samples, or samples derived from cell cultures in tissue flasks, e.g. HeLa cells. As mentioned above, it is also possible to lyse certain samples or tissues (e.g. bacterial or plant cells) using conventional lytic agents and subsequently subject the lysed sample to incubation with a composition of the present invention. The incubation of the sample in the composition may take place at room temperature, below room temperature, e.g. at 4° C., or in above room temperature, e.g. in an incubator or heating device. An increase in temperature generally accelerates the lytic process. In the incubation process, proteins, polypeptides, complex carbohydrates, cytoplasm, etc. dissolve completely in the composition of the invention.

The compositions of the present invention function particularly well when the water concentration does not exceed 20% (v:v) and about 2% (m:m) ammonium acetate. Higher water concentrations can be used, but this would result in a less efficient lysis and loss of RNA. One reason therefore is that an increased water content results in increased solubility of RNA in HFIP. Another reason is that cell lysis and protein solubilization is incomplete with higher water concentrations. In the present compositions, a water content of about 10% (v:v) is preferred. In a further preferred embodiment the compositions of the invention comprise about 10% (v:v) H₂O and up to 20% (v:v) of 2-propanol.

It is possible to add a matrix material to the suspension, wherein the matrix material has the capacity to bind nucleic acids, e.g. silicium dioxide. Addition of this material makes subsequent separation steps, e.g. by centrifugation easier and more efficient.

After the incubation step of the cell sample in the composition of the invention, the suspension is subjected to a step that separates nucleic acids from other components originally present in the sample material. DNA and RNA are insoluble in HFIP and are separated from the suspension by suitable procedures, such as centrifugation, filtration, or any other way of separation. DNA and RNA precipitation by centrifugation is more efficient when silicium dioxide, e.g. in an amount of 1-10% (m:m), e.g. 1%, 2.5%, 5%, 7.5% or 10% is added either to the suspension or to the original composition of the invention before addition of the cell sample. The centrifugation step may be performed as in other conventional nucleic acid purification methods, e.g. using a microcentrifuge, at 4° C. or room temperature, e.g. at about 10,000-25,000 xg for about 30 seconds to 15 minutes, e.g. 5 minutes at 21,000 xg. Precipitation (also referred to as “sedimentation”) of nucleic acids by centrifugation is more efficient when about 20% (v:v) of 2-propanol is added to the obtained suspension comprising the inventive composition and the cell sample.

Subsequently, the precipitated or sedimented nucleic acids are separated from the remaining suspension by decanting, aspirating or evaporation of the supernatant. Due to the low boiling point of HFIP (58° C.) said compound evaporates relatively fast. It is possible to perform a washing step of the precipitated nucleic acids, e.g. using once again the compositions of the invention, 20% 2-propanol, pure HFIP without additional ingredients, ethanol, or similar washing solutions. The washing solution is once again removed by aspiration, decantation, filtration or evaporation.

Thereafter, the nucleic acids are dissolved in water, e.g. DEPC-treated water. Whereas RNA dissolves completely in water, DNA remains precipitated, but may later, once the dissolved RNA is removed, be removed in a suitable buffer, e.g. in high-molar solutions of ammonium or sodium salts. It was surprisingly noted that the obtained RNA following the methods of the invention was of extremely high purity and that the integrity was beyond expectation.

The obtained RNA or DNA may be subjected to further analysis steps, e.g. reverse transcription, amplification (e.g. by PCR, qPCR, etc.), hybridization (e.g. Northern Blotting, FISH), sequencing, e.g. Next Generation Sequencing.

The above method can be performed by individuals, e.g. staff in a diagnostic laboratory or research laboratory, or in a semi-automated or automated laboratory platform, e.g. as obtainable from diverse suppliers, e.g. Vela Diagnostics (Singapore), or Eppendorf GmbH (Hamburg, Germany) or any other adequate commercial supplier.

In an alternative method aiming at the analysis of polypeptides or proteins (proteomics), the supernatant of the suspension comprising the sample and the compositions of the present invention is stored for further analysis. It is an advantage of the methods and tools that the cellular proteome is present in the suspension and that the suspension is free of detergents or chaotropic substances that have to be removed carefully, frequently accompanied by substantial losses of proteinaceous material, before further analysis of the proteome. As indicated above, HFIP has a very low boiling point so that the evaporation of the composition is easily accomplished at temperatures which do not severely damage proteins. It is of course also possible to wash the obtained proteins after evaporation of the composition to remove low molecular weight proteins. It is also possible to separate the proteins by precipitation, e.g. using centrifugation steps after the separation of the protein contents from the nucleic acids, or after optional washing steps.

HFIP is a toxic substance, but it may be recycled after usage and can be reused in industrial processes, even in processes or laboratories for the analysis of nucleic acids or proteins.

Kits of the present invention, which can be stored at room temperature and shipped under conditions that do not require specific attention, e.g. cooling, etc., usually comprise instructions for use, positive controls (e.g. a given nucleic acid that is optionally present in cells, e.g. transformed cell lines and the like), at least one compositions of the invention, optionally washing buffers, buffers to dissolve nucleic acids, matrix material for specifically binding of nucleic acids, e.g. silicium dioxide, plastic containers, or any other material that can be used in the isolation of nucleic acids or proteins.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

The following examples illustrate the invention, but should by no means be construed as limiting the same.

### EXAMPLES

### Example 1

### HeLa Cell Lysis with HFIP Lysis Buffer

76 mg of a frozen HeLa cell pellet (HeLa cell mass RELIATech GmbH) with an estimated protein content of about 1.9 mg were lysed and homogenized with 2000 μl HFIP comprising 5 wt.-% ammonium acetate and 10 Vol.-% (v:v) water. The resulting mixture was shaken vigorously for one minute and centrifuged for 5 minutes at 21,000 xg at a temperature of 4° C. Subsequently, two phases were formed—a liquid organic phase and a solid precipitate. Proteins, lipids and metabolites remained in the liquid organic phase while DNA and RNA formed the solid precipitate.

The liquid phase was pipetted off and transferred into a fresh tube. The remainder of the liquid phase covering the precipitate was evaporated to dryness. The nucleic acid precipitate was washed once with pure HFIP, centrifuged for one minute at 21,000 xg. Thereafter, the pellet was once again air-dried for about 15 minutes. The liquid organic phase containing proteins, lipids and metabolites was subjected to evaporation to obtain a protein pellet. The pellet was redissolved in 382 μl of 4M guanidinium chloride to a final protein concentration of about 5 g/1. Protein cystines were reduced by DTT and cysteines alkylated by iodacteamide following standard procedures. To solubilize optionally present HFIP insoluble protein, the nucleic acid pellet can be treated with the same volume of guanidinium chloride, the same amounts of DTT and iodoactamide for cysteine reduction and alkylation. 20 μl of each solution were precipitated with icecold acetone and redissolved in 20 μl of 0.5% LDS buffer (Invitrogen) for 1D-PAGE analysis. Thereafter, 10 μl of each sample were subjected to LDS 1D-PAGE analysis applying the NuPAGE® Bis-Tris mini gels system and MES running buffer (Invitrogen) in combination with Coomassie staining.

### Results:

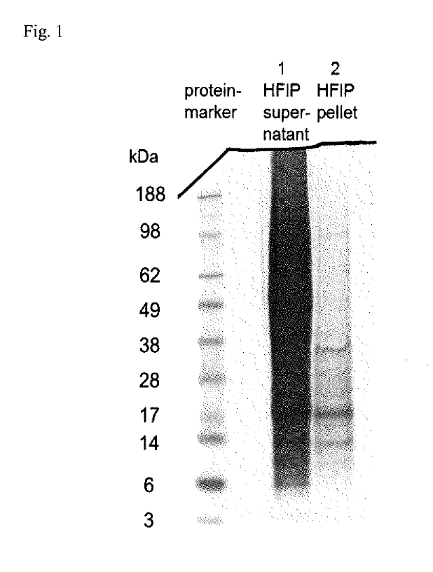

HeLa cells are lysed completely in concentrated organic solvent HFIP containing 5 wt.-% ammonium acetate and 10 Vol %-% (v:v) water. As demonstrated in FIG. 1, total cellular protein is completely dissolved in HFIP buffer (lane 1). Only traces of protein carryover also visible as high abundant protein bands in the supernatant lane were detectable in the HFIP pellet (lane 2). This shows that highly concentrated HFIP comprising an organic salt and low amounts of water is an excellent cell lysing agent. Whereas nucleic acids are insoluble in HFIP, the total cellular proteome and small organic and inorganic molecules are completely dissolved.

### Example 2

### Cell Membrane Protein Solubility in HFIP Lysis Buffer

A frozen HeLa cell pellet of 10⁷ cells (Calbiochem) with a total protein content of 8.5 mg was processed according to the protocol of the Proteo Extract Subcellular Proteome Extraction Kit (Calbiochem) to extract only membrane proteins from the cells. The membrane protein fraction 2 contained 1.7 mg of the membrane subproteome in total. Aliquots of 100 μg membrane protein were treated by chloroform/methanol protein precipitation according to the method of Wessel/Flugge, (cf. Anal Biochem. 1984 April; 138(1):141-3).

Desiccated pellets were either redissolved in 100 μl HFIP containing 5 wt.-% ammonium acetate and 10 Vol.-% water or dissolved in 100 μl RIPA cell lysis buffer. Both samples were centrifuged for five minutes at 4° C. at 21,000 xg. 40 μl of the HFIP membrane protein solution were evaporated to dryness. The membrane protein solution in RIPA buffer was processed once more according to the Wessel/Flugge protocol for protein separation and clean up. Both, HFIP and RIPA protein pellets were dissolved in LDS sample buffer and 40 μg protein of each sample were directed to LDS 1D-PAGE analysis using the NuPAGE Bis-Tris mini gels system and MES running buffer from Invitrogen applying silver staining.

### Results

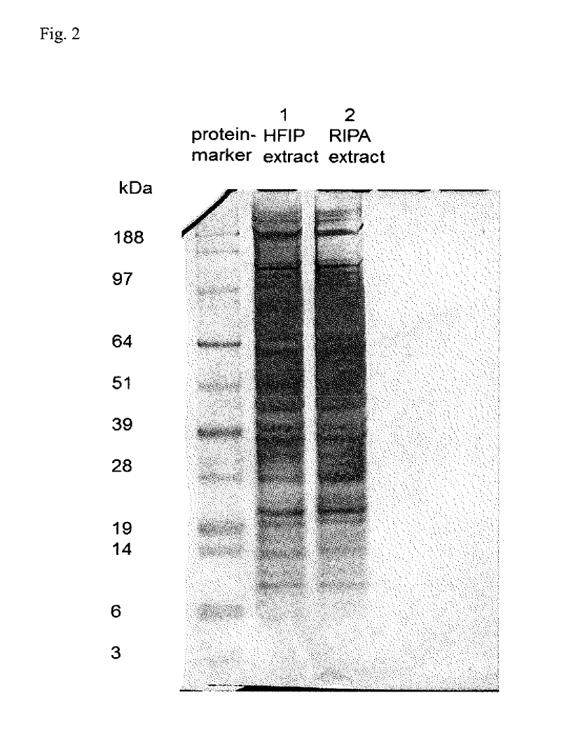

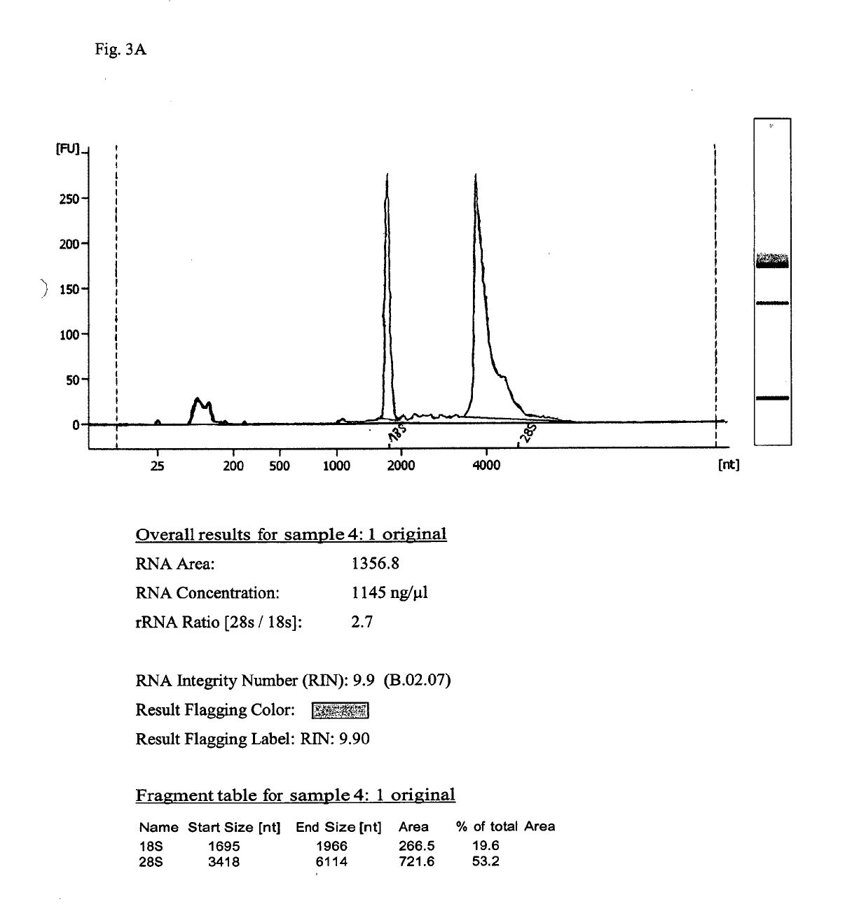

Both proteomes, obtained with the HFIP lysis buffer and the RIPA buffer treated membrane protein pellets, respectively, displayed the same complex pattern on 1D-PAGE as demonstrated in FIG. 2. Unexpectedly, almost pure HFIP is an excellent dissolver of complex and difficult to manage membrane proteomes. This demonstrates the excellent protein solubilization capacity in total cell lysis. HFIP separated proteomes can be used directly in further protein analysis techniques.

### Example 3

### RNA Isolation with HFIP Lysis Buffer

Cell lysis and RNA isolation experiments were performed by subjecting 74, 82 and 88 mg (samples 1-3) of frozen HeLa cells (HeLa cell mass RELIATech GmbH) to 800 μl of HFIP lysis solution containing 0.25 wt.-%, 2.5 wt.-%, and 5 wt.-% ammonium acetate, respectively. Complete homogenization was achieved by vigorously mixing the samples on a lab vortexer for 2 minutes. Nucleic acids were precipitated by centrifugation at room temperature for 5 minutes at 21,000 xg. Supernatants were pipetted off. The DNA/RNA pellets were washed once with pure 99% HFIP to remove residual proteinaceous material and salts and thereafter sedimented again by centrifugation and the remaining pellet was dried at room temperature. RNA was solubilized in 300 μl DEPC water.

### Results:

345 μg, 317 μg and 343 μg, respectively, of total RNA were extracted from HeLa samples 1, 2 and 3 as determined by Nanodrop analysis. In parallel, the Agilent 2100 Bioanalyser Eukaryote total RNA nanochip analysis was used to determine the RNA quantity, which resulted in amounts of 448 μg, 365 μg and 378 μg, respectively.

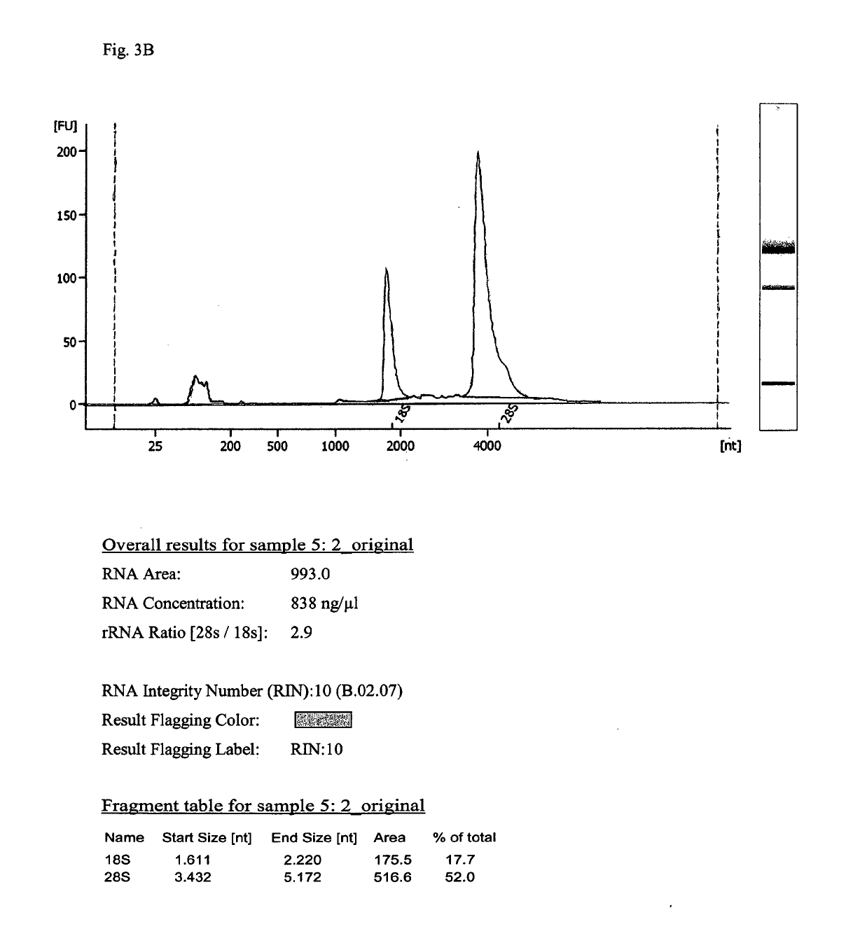

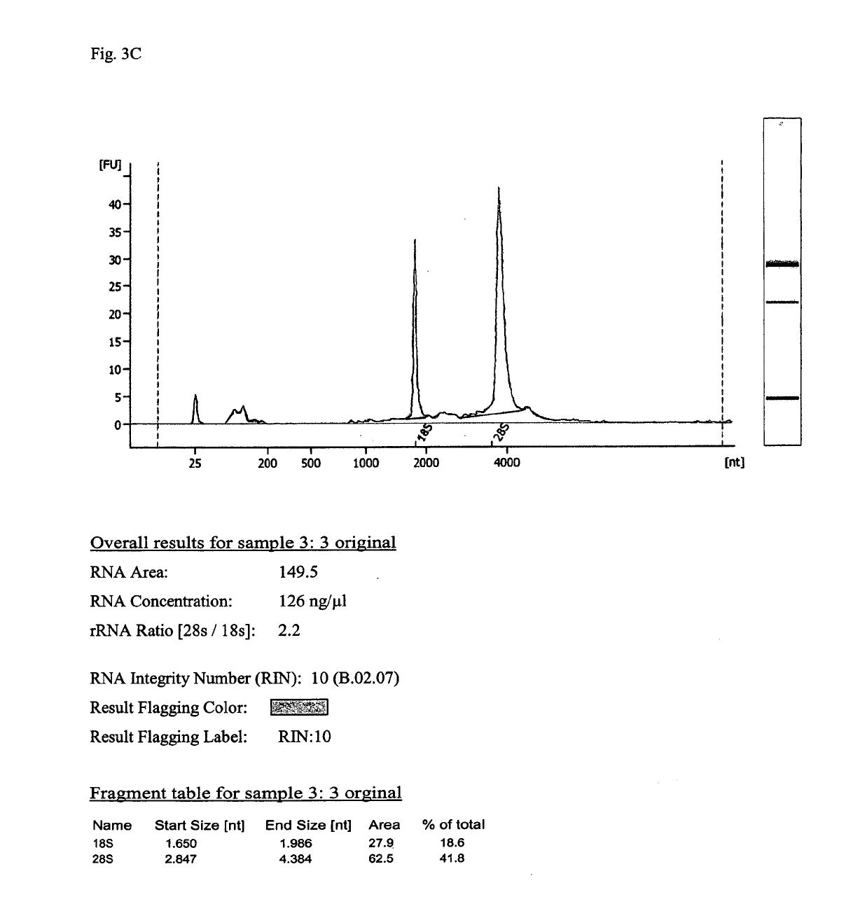

The RNA isolated from three HeLa samples was intact and showed no visible signs of degradation, as determined by quantification of ribosomal 18S and 28S RNA ratios of 9.9, 10.0 and 10.0, respectively, taking the RNA integrity factor (RIN) as parameter (cf. FIG. 3). Protein contaminations in the RNA samples are not detectable as determined using the Nanodrop UVA.sub.260/280 ratios which were 1.96, 2.04 and 1.83, respectively.

These results indicate a lack of protein contaminations. A high quantity of high quality RNA was isolated. Additional protection against RNAse degradation is not necessary when RNA is extracted with highly concentrated HFIP solutions according to the invention, because proteins in solution are completely denatured.

DNA is completely insoluble and denatured after treatment either in pure HFIP, HFIP comprising ammonium acetate, or in HFIP comprising ammonium acetate and low amounts of water.

### Example 4

### HFIP Separated RNA is Substantially Free of DNA

Cell lysis of 5 samples of 50 mg frozen HeLa cells (HeLa cell mass RELIATech GmbH) was subjected to 500 μl HFIP composition comprising 2 wt.-% ammonium acetate and 0 Vol.-%, 2.5 Vol.-%, 5 Vol.-%, 10 Vol.-% and 20 Vol.-% (each v:v), respectively, of water.

Samples were homogenized by vigorously shaking the samples on a lab vortexer for 2 minutes. Nucleic acids were precipitated by centrifugation for 5 minutes at 21,000 xg. The supernatant was simply decanted. The precipitate was washed once with pure HFIP and thereafter separated for 5 minutes at 21,000 xg and the supernatant was decanted. The remaining supernatant covering the pellet was evaporated for 15 minutes at room temperature until the pellet was dry. RNA was solubilized with 100 μl DEPC water. Subsequently, RNA was digested with RNase. DNA concentration was determined using the Bioanalyzer 2100 and DNA LabChip 25-1000 bp.

### Results:

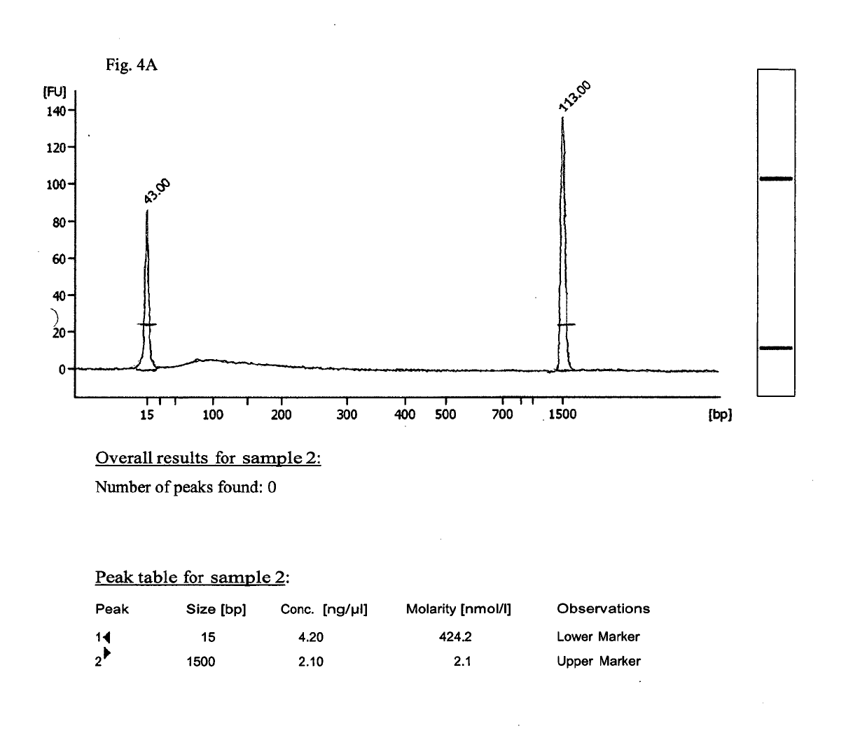

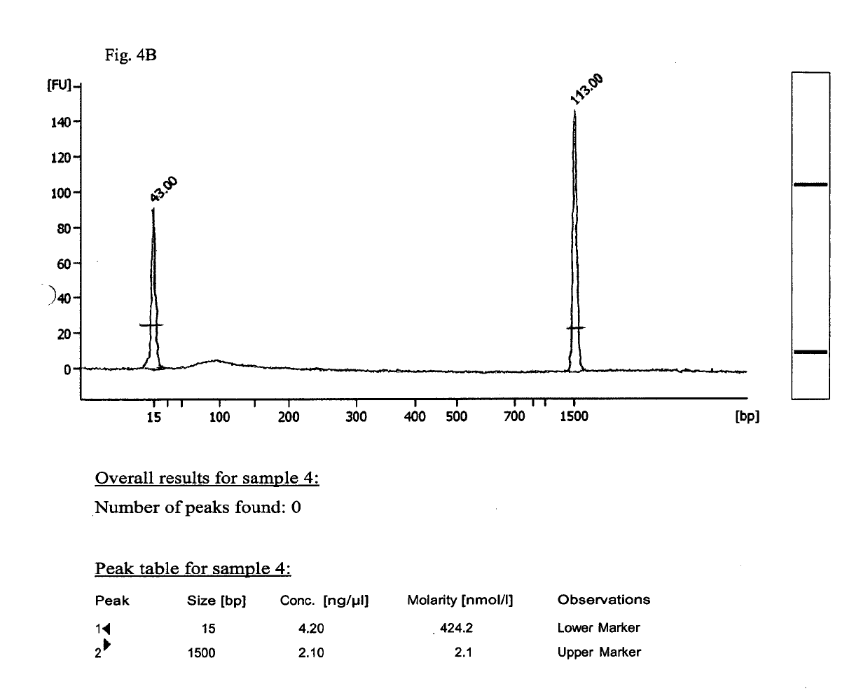

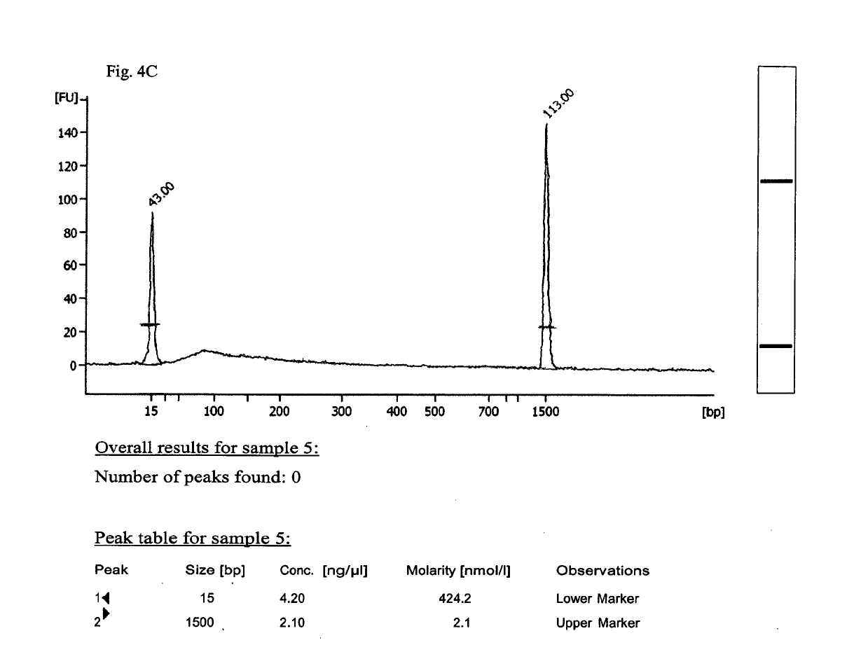

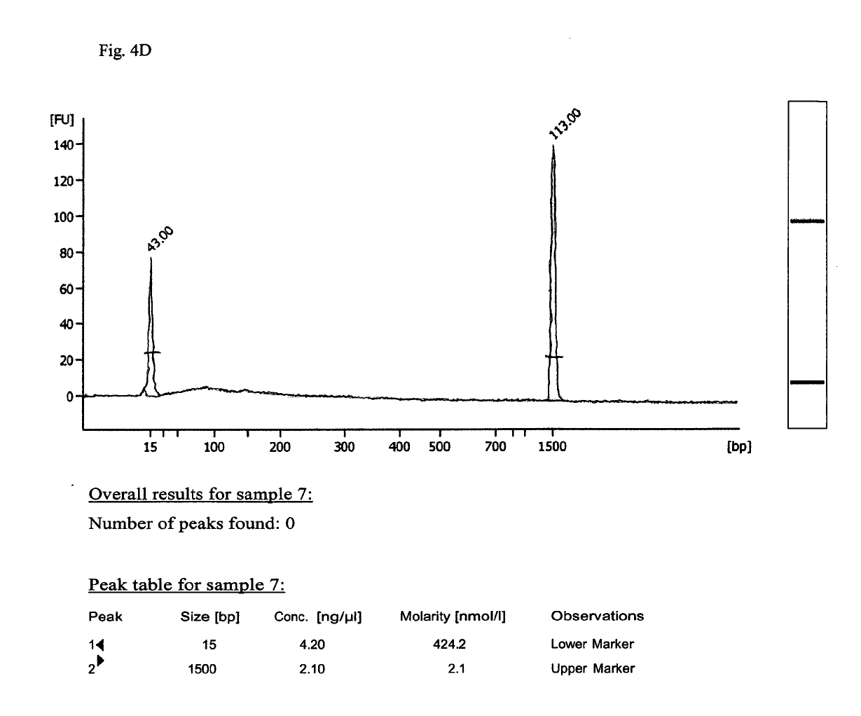

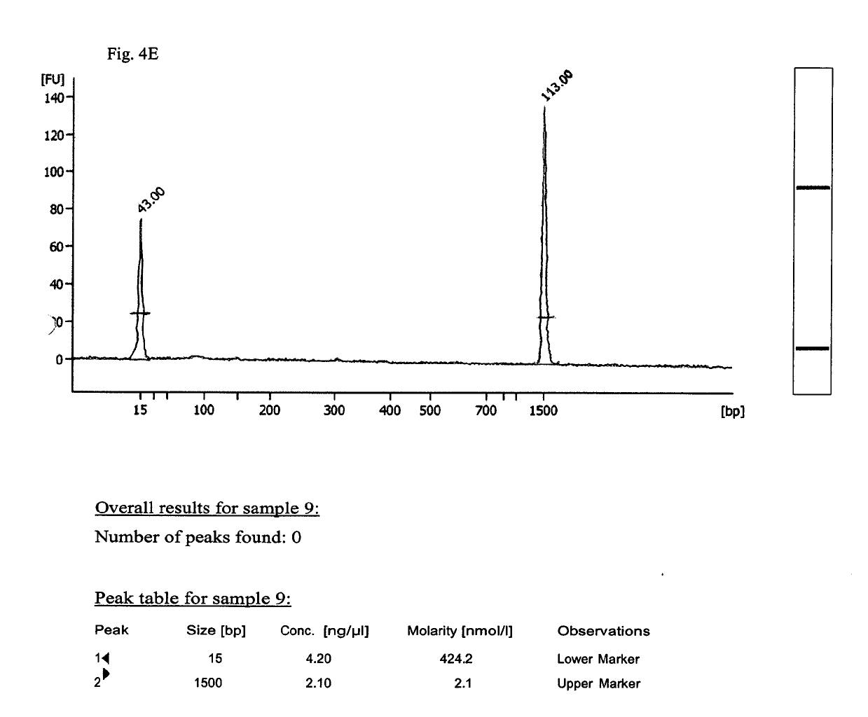

All samples were absolutely free of DNA as depicted in FIG. 4. Further, up to a content of 20 Vol.-% (v:v) water in the compositions of the invention, no DNA is detected in the RNA samples. This shows that nucleic acid pellets obtained after treatment of samples with HFIP lysis buffer and subsequent separation using centrifugation is substantially free of DNA. Further, HFIP lysis buffer comprising 2 wt.-% ammonium acetate and up to 20 Vol.-% water denatures DNA so that it is completely insoluble in water. This is an advantage in any method relying exclusively on the analysis of RNA without potentially disturbing DNA.

### Example 5

### HFIP Lysis Buffer Separated RNA can be Used for cDNA Amplification

Total RNA extraction for RT-PCR analysis was performed. The HeLa cell samples listed below were subjected to lysis in 500 μl HFIP lysis buffer comprising 95% HFIP and 5% ammonium acetate:
1) 66 mg frozen HeLa cells;
2) 45 mg of frozen HeLa cells (both 1) and 2) obtained from HeLa cell mass RELIATech GmbH), and
3) HeLa pellet of 10⁷ cells (Calbiochem).

Additionally, the HeLa total RNA standard (sample 4) in an Invitrogen Superscript Kit was used for RT-PCR analysis.

Samples were homogenized by vigorously shaking on a lab vortexer for 2 minutes. Nucleic acids were precipitated by centrifugation for 5 minutes at 21,000 xg at room temperature. The supernatant was pipetted off and the precipitates were resuspended once with pure HFIP followed by centrifugation for one minute at 21,000 xg. The remaining supernatants covering the nucleic acid pellets were evaporated for 15 minutes at room temperature. Subsequently, RNA was solubilized in 300 μl DEPC water.

Thereafter, cDNA synthesis from total RNA extracts of samples 1-4 was performed using the SuperScript Double-Stranded cDNA Synthesis Kit (Invitrogen). cDNA synthesis was conducted with 25 μg of total RNA extract.

Thereafter, PCR amplifications were performed using 50 ng of cDNA and 26 pmol primers specific for DNA replication licensing factor-5 (MCM5_Exon8), glyceraldehyde-3-phosphate dehydrogenase-1 and -2 (GAPDH1_Exon8, GAPDH2_Exon8). PCR products were analyzed by agarose gel electrophoresis and ethidium bromide staining to visualize the amplification products.

### Results:

Total RNA yields isolated from HeLa cells pellet samples 1-3 were 195, 384 and 390 μg as determined by Nanodrop analysis. The UV 260/230 absorption ratios were >2.0 for each sample. Accordingly, RNA samples were free of protein contaminations.

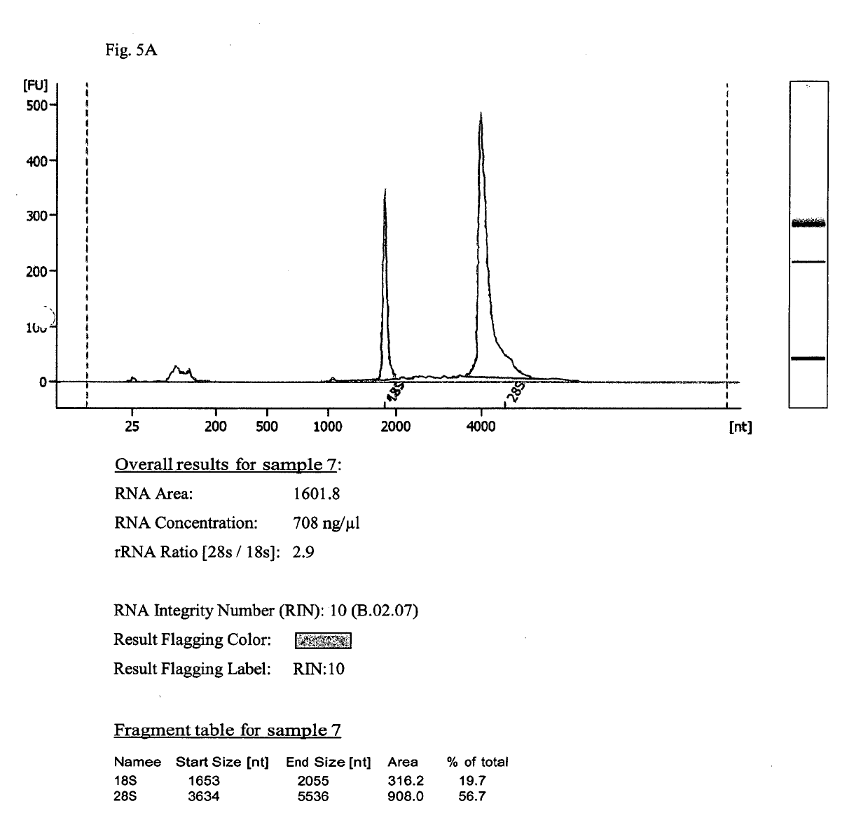

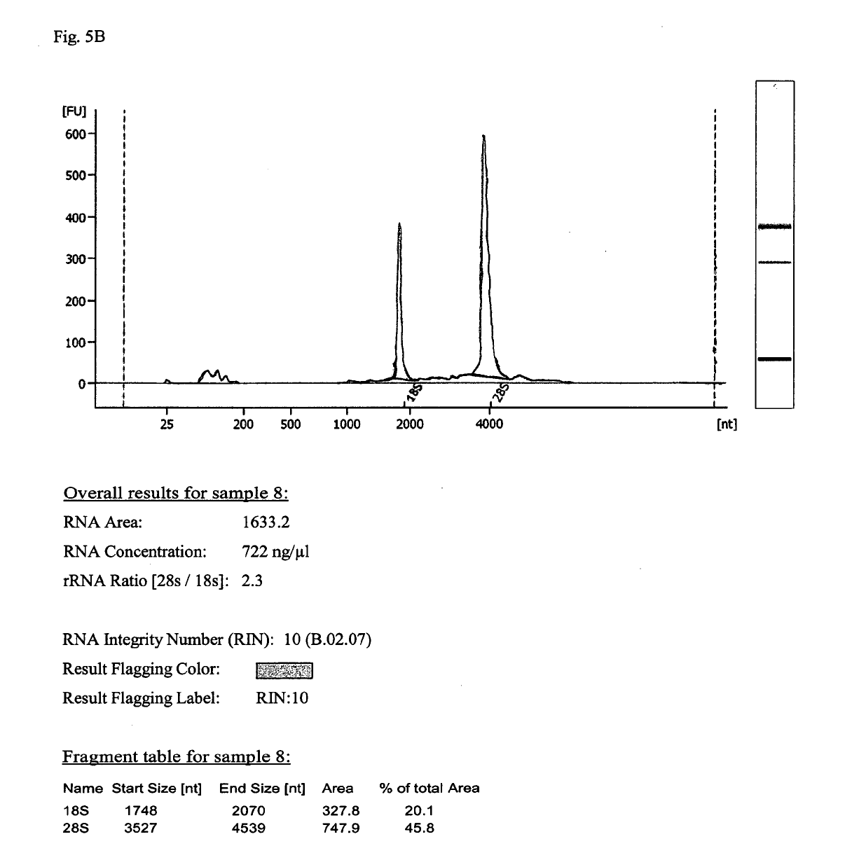

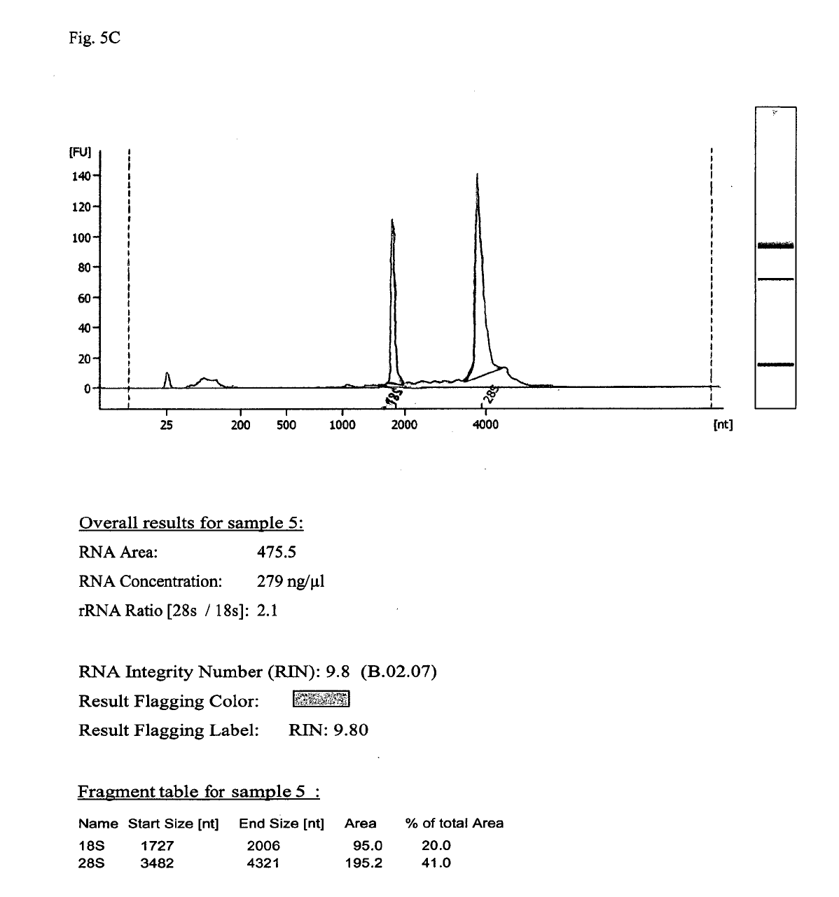

RIN factors of 10 for RNA samples 1, 2 and 3 and 9.8 for the Super Script HeLa RNA control sample were measured using the Agilent 2100 Bioanalyzer Eukaryote total RNA nano-chip analysis as shown in FIG. 5A-D. According to these results, high yields of pure and intact total RNA samples are obtained using the HFIP lysis buffer compositions according to the invention.

cDNA was successfully transcribed from total mRNA isolated using the above HFIP lysis buffer. Comparable cDNA concentrations were obtained for samples 1-3 with 337, 556 and 525 ng/μl cDNA. In the Superscript control sample 4, 198 ng cDNA were detected.

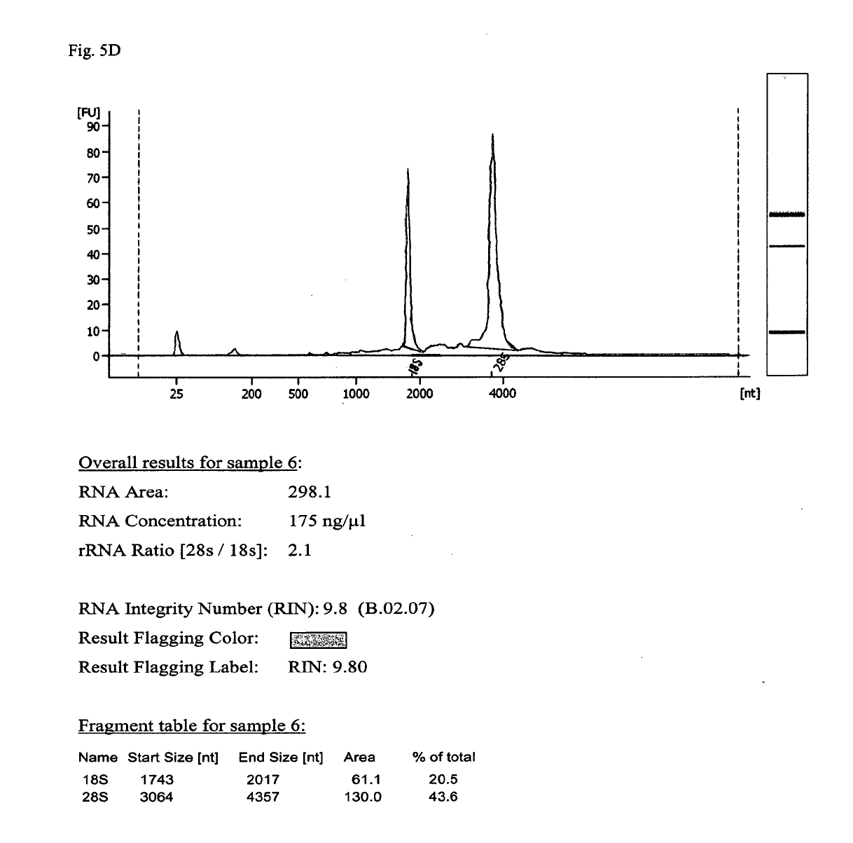

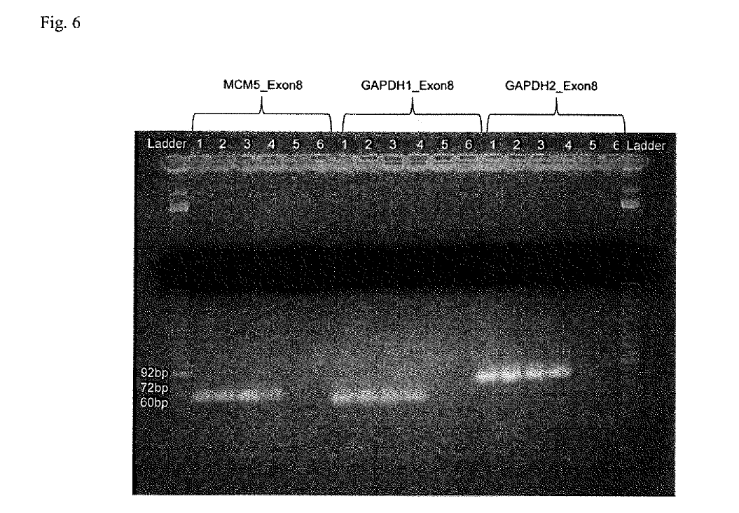

PCR amplifications of MCM5_Exon8, GAPDH1_Exon8, GAPDH2_Exon8 were successfully performed with total RNA samples 1-3 obtained with the inventive HFIP lysis buffer. The intensities in lanes 1-3 on the agarose gel were about the same as the Invitrogen Superscript HeLa RNA standard sample in lane 4 for each amplified cDNA as depicted in FIG. 6.

No amplification products were observed with two water controls as shown in lanes 5 and 6 of each cDNA primer used for PCR analysis. These results indicate that total RNA preparations isolated with HFIP lysis buffer according to the invention are extremely pure so that they can be used for cDNA synthesis and PCR amplification.

### Example 6

### microRNA Isolation with HFIP Lysis Buffer

Four frozen T-lymphocyte cell pellet samples extracted from human blood containing about 5.0×10⁶ cells were thawed and washed once with PBS buffer. Pelleted intact cells were lysed with 500 μl of a composition comprising HFIP, 2 wt.-% ammonium acetate and 10 Vol.-% (v:v) water. The obtained suspension was homogenized by shaking for 2 minutes using a lab vortexer. Nucleic acids were sedimented by centrifuging for one minute at 21,000 xg. The supernatant containing proteins was decanted.

RNA/DNA pellets were dried for 10 minutes using a Speed Vac concentrator. RNA was dissolved in 50 μl DEPC water. RNA sample concentrations were determined using a Nanodrop UV photometer.

For microRNA (miRNA) expression analysis 100 ng of total RNA was reverse-transcribed using the miRCURY LNA™ Universal RT kit according to the instructions of the manufacturer (Exiqon A/S, Vedbaek, Denmark).

The expression of miRNA species was detected by PCR using the miRCURY LNA Universal RT microRNA PCR LNA PCR primer sets hsa-miR-146a, -155, -223, -326 and the reference gene PCR primer set SNORD44 (hsa) (Exiqon A/S, Vedbaek, Denmark). The PCR mixture contained 200 μM of each dNTP, and LNA PCR primer set according to manufacturer's instruction, 0.025 IU of polymerase (BIOTAQ DNA Polymerase, Bioline GmbH) and 1× concentrate of fluorogenic substrate Evagreen Dye (BIOTIUM, Hayward, Calif., USA). PCR was performed in triplicate at 95° C. for 10 minutes followed by 45 cycles at 95° C. for 10 seconds and 60° C. seconds followed by a melting analysis on the LightCycler 480 System (Roche, Mannheim, Germany). The expression of miRNA was calculated semi-quantitatively by the ΔΔCt-method. All results are presented as relative expression to SNORD44 reference gene expression (Table 1).

### Results:

RNA quality control was performed using a Nanodrop UV spectrophotometer. Total RNA concentrations of samples 1-4 were 693, 519, 419 and 682 ng/μl, respectively.

mRNA was successfully transcribed into cDNA. Selected microRNA targets miR-146a, -155, -223, -326 and the reference gene SNORD44 were quantified by RT-qPCR amplification in all 4 samples, see Table 1.

The RT-qPCR analyses of T-lymphocyte microRNA levels shows that the methods of invention yield total RNA including functional high quality small microRNAs.

TABLE 1
Expression of miRNA as calculated semi-quantitatively by
the ΔΔCt-method. All results are presented as relative
expression to SNORD44 reference gene expression.

| Sample No. | miR-146a | miR-155 | miR-223 | miR-326 | SNORD44 |
|---|---|---|---|---|---|
| 1 | 25.5 | 27.0 | 22.0 | 28.8 | 25.1 |
| 2 | 29.4 | 31.7 | 27.2 | 30.1 | 30.5 |
| 3 | 26.9 | 28.9 | 23.6 | 28.4 | 27.5 |
| 4 | 33.4 | 34.8 | 27.9 | 32.1 | 30.7 |
| mean | 28.8 | 30.6 | 25.2 | 29.9 | 28.5 |
| Standard | 3.5 | 3.4 | 2.8 | 1.7 | 2.7 |
| deviation | | | | | |

1. A composition for isolating nucleic acids comprising:
(a) at least 70% (v:v) 1, 1, 1, 3, 3, 3, hexafluoro-2-propanol (HFIP), and
(b) a salt, and/or
(c) an acid, and/or
(d) a base, and/or
(e) water in an amount of 1.0 to 20.0 Vol.-%,
wherein the organic salt, the organic acid, inorganic acid, organic base, and/or inorganic base is added to the HFIP in an amount of 0.05 weight-% up to saturation level.

2. (canceled)

3. The composition according to claim 1, wherein the salt is selected from the group comprising ammonium, sodium, potassium, lithium, manganese, and barium, or a salt of an organic acid comprising formic acid, acetic acid, propionic-acid, long chain fatty acids, di-carbonic acids comprising oxalic-, malonic-, succinic-, and citric-acid.

4. (canceled)

5. (canceled)

6. (canceled)

7. (canceled)

8. A method of extracting nucleic acids from a sample comprising the steps:
(a) providing a cell sample,
(b) adding a composition according to claim 1 to said sample,
(c) suspending the cell sample in said composition,
(d) separating nucleic acids,
(e) dissolving nucleic acids in an aqueous buffer.

9. The method of claim 8, wherein the separation of nucleic acids is performed by centrifugation of the suspension and precipitation of the nucleic acids to provide a supernatant, and a nucleic acid sediment, or by filtration of the nucleic acids.

10. The method of claim 8, further comprising adding at least one of aliphatic alcohols selected from the group comprising methanol, ethanol, 1-propanol, 2-propanol, n-butanol, isobutanol, and 2-butanol, isomers of amyl alcohol, trifluoroethanol, or esters of aliphatic alcohols and fatty acids selected from the group comprising methylacetate, ethylacetate, or ketones selected from the group dimethyl ketone, methyl ethyl ketone, or ethers selected from the group comprising diethyl ether, 1, 2-, 1, 3-, 1, 4-dioxane, or other organic solvents comprising acetonitrile, and chloroform, prior to or after step (b).

11. The method of claim 10, wherein the at least one of aliphatic alcohols selected from the group comprising methanol, ethanol, 1-propanol, 2-propanol, n-butanol, isobutanol, and 2-butanol, isomers of amyl alcohol, trifluoroethanol, or esters of aliphatic alcohols and fatty acids selected from the group comprising methylacetate, ethylacetate, or ketones selected from the group dimethyl ketone, methyl ethyl ketone, or ethers selected from the group comprising diethyl ether, 1, 2-, 1, 3-, 1, 4-dioxane, or other organic solvents comprising acetonitrile, and chloroform is added in an amount of 0.1 to 25.0 Vol.-% of the suspension.

12. The method of claim 10, wherein further a nucleic acid binding material is added to the suspension obtained in step (c).

13. The method of claim 8, wherein the supernatant is removed by decanting, aspiration or evaporation.

14. The method of claim 8, wherein the nucleic acid sediment is washed with a solution comprising at least 75%, at least 80% HFIP, preferably at least 90% HFIP, more preferably at least 95% HFIP, or 99% HFIP, wherein said washing solution is removed after the washing step.

15. The method of claim 8, wherein the nucleic acid is RNA.

16. The method of claim 8, wherein the nucleic acid is mRNA or microRNA.

17. The method of claim 8, wherein the aqueous buffer in step (e) is sterilized water or DEPC treated water.

18. The method of claim 8, wherein the RNA is subjected to reverse transcription.

19. The method of claim 8, wherein the nucleic acid is subjected to amplification, hybridization, or sequencing.

20. A kit for the lysis of a cells present in a sample comprising a composition according to claim 1 and instructions for use.

21. The kit according to claim 20 further comprising at least one of aliphatic alcohols selected from the group comprising methanol, ethanol, 1-propanol, 2-propanol, n-butanol, isobutanol, and 2-butanol, isomers of amyl alcohol, trifluoroethanol, or esters of aliphatic alcohols and fatty acids selected from the group comprising, methylacetate, ethylacetate, or ketones selected from the group dimethyl ketone, methyl ethyl ketone, or ethers selected from the group comprising diethyl ether, 1, 2-, 1, 3-, 1, 4-dioxane, or other organic solvents comprising acetonitrile, and chloroform and/or a nucleic acid binding material.
